(19) United States
(12) Patent Application Publication
(10) Pub. No.: US 2023/0414918 A1
(43) Pub. Date: Dec. 28, 2023

(54) CONNECTION SYSTEM FOR A DRUG DELIVERY DEVICE

(71) Applicant: West Pharmaceutical Services, Inc., Exton, PA (US)

(72) Inventors: Zachary Wallace, Philadelphia, PA (US); Lawton Edward Laurence, Chester Springs, PA (US); Menaka Abeygunawardena, Blue Bell, PA (US); Aidan Hyde, Collingswood, NJ (US); Andrew Mazzotta, Grand Rapids, MI (US); Richard James Brough, Scottsdale, AZ (US); Spencer W. Pratt, Glendale, AZ (US); Nolan P. Walker, Tempe, AZ (US); Michael J. Sullivan, Birdsboro, PA (US)

(21) Appl. No.: 18/037,305

(22) PCT Filed: Nov. 16, 2021
(86) PCT No.: PCT/US2021/059533

§ 371 Date: May 16, 2023

Related U.S. Application Data
(60) Provisional application No. 63/114,250, filed on Nov. 16, 2020.

(51) Int. Cl. A61M 39/16; A61M 5/162

(52) U.S. Cl. CPC A61M 39/16; A61M 5/162

(57) ABSTRACT

Connection systems for injection devices are disclosed. Also disclosed are injection devices and methods of using the same. A connection system includes a first and second connection assembly and a coupling assembly to secure the same. The first connection assembly connects to a medicament container, and the second connection assembly has a needle movable toward the medicament container. Removable flexible barriers are disposed on the first and second connection assemblies and can be removed once the connection assemblies are coupled together to form an aseptic connection between the two assemblies. The needle can be moved toward the medicament container to pierce a septum on the container to form a fluid communication between the container and the needle.

### CROSS REFERENCE TO RELATED APPLICATIONS

This application claims the benefit of U.S. Provisional Application No. 63/114,250, filed Nov. 16, 2020, the entirety of which is incorporated herein for any and all purposes.

### TECHNICAL FIELD

The present disclosure relates to a connection system for creating an aseptic connection between a container and a delivery conduit of an injection device, to a container for creating an aseptic connection with an injection device, to a connection assembly for creating an aseptic connection with a container, and to associated methods.

### BACKGROUND

Injection devices, such as syringes and autoinjectors, deliver medicaments from a medicament container through a hypodermic needle. Because the hypodermic needle delivers medicaments to a patient's body, sterility of injection devices and medicament containers is of utmost importance.

Often, injection devices are manufactured and sold separately from medicament containers. As a result, a single injection device can be used to deliver a wide range of treatments, by connecting the injection device to a medicament container containing a medicament type and dosage for any one of the wide range of treatments. Before use, a user (medical professional, or patient) couples a medicament container containing a type and dose of medicament for the treatment required, to the injection device. A fluid connection is formed between the container and the hypodermic needle of the injection device, such that injection of the medicament can be performed. Collectively, the injection device and the container form an injection system.

In many injection systems, the container is initially sealed by a septum, thereby sealing the medicament within the container and maintaining sterility of the medicament. Furthermore, the injection device includes a transfer needle (separate from the hypodermic needle), the transfer needle being configured to pierce the septum once the container and the injection device are connected. A fluid conduit fluidly connects the transfer needle to the hypodermic needle. Therefore, once the transfer needle has pierced the septum, the medicament can be delivered to a patient through the hypodermic needle. Such injection systems are particularly useful for home use because the connection between the container and the injection device is easy to achieve (by simply pushing the container or transfer needle into place). Such injection systems can also be used in hospitals.

However, a problem exists that sterility of the outer surface of the septum, and of the transfer needle, is difficult to ensure. Accordingly, there is a risk that one or both of the outer surface of the septum and the transfer needle become contaminated, and that this contamination could enter the patient when an injection is formed. This risk is exaggerated where injection systems are used in the home, because contamination is more likely to occur where a medical professional does not operate the injection system. Furthermore, a user may keep an injection device and containers in a drawer or cupboard in the home, where contamination is likely.

### SUMMARY

Various exemplary aspects are described below. Each individual aspect can represent a single aspect of the disclosure, or, alternatively, can be included (either wholly or in part) in one or more aspects. Each aspect can be a stand-alone aspect or be intended to interact with, or be combined with, one or more of any of the other aspects below or otherwise described throughout this application in a preferred combination.

In a first aspect, there is provided a connection system for creating an aseptic connection between a container (e.g., a medicament container) and a delivery conduit of an injection device. The delivery conduit can include a transfer needle. The connection system comprising:
a first connection assembly comprising:
   a first engagement body comprising a first end having a first opening, the first end configured to form a seal with a septum of a container, a second end having a second opening, a first cavity between the first and second openings, and a first engagement surface at the second end of the first engagement body; and
   a first flexible barrier arranged to seal the second opening;
a second connection assembly comprising:
   a second engagement body comprising a first end having a third opening, a second end having a fourth opening, a second cavity between the third and fourth openings, and a second engagement surface at the second end of the second engagement body;
   a needle operably connected to the second engagement body; and
   a second flexible barrier arranged to seal the fourth opening;
wherein the first engagement surface further comprises a first sealing interface configured to form a seal with a second sealing interface on the second engagement surface; and
wherein the connection system is configured such that the needle is moveable relative to the fourth opening of the second engagement body from a first position where the needle is spaced from the third opening of the second engagement body, to a second position where the needle extends through the third opening. For example, the needle may be moveable from a first position where the needle is spaced from the third opening to a second position where the needle extends through the third opening and into the first cavity.

Because of the provision of the first flexible barrier, contaminants cannot enter the first cavity via the second opening. Therefore, when a container having a septum is coupled with the first connection assembly such that the septum forms a seal with the first end, the first flexible barrier prevents contaminants from reaching the septum via the second opening. An aseptic environment can thereby be maintained around the septum.

Similarly, because of the provision of the second flexible barrier, contaminants cannot reach the needle via the fourth opening. An aseptic environment can thereby be maintained around a portion (e.g., a free end) of the needle that is located within the second cavity. That is to say, the second cavity may comprise an aseptic cavity.

A user may couple the first and second connection assemblies to one another such that the first sealing interface forms a seal with the second sealing interface, and subsequently remove the first and second flexible barriers, to thereby form a single aseptic area that contains the septum and the needle. Advantageously, though an aseptic connection is formed at this time, a fluid connection between the feature attached to the first connection assembly and the feature attached to the second connection assembly is not yet formed. This prevents the fluid from leaking out of the connection system or otherwise becoming contaminated.

Upon user activation, the needle may be moved into the first cavity to pierce the septum, thereby establishing an aseptic fluid pathway from the container to the needle. Sterility of the system is thus maintained throughout transport and through fluid connection. A method of forming such an aseptic fluid pathway is discussed in further detail in the sixth and seventh aspects.

In a second aspect there is provided a container (e.g., medicament container) for creating aseptic connection with an injection device, the container sealed by a pierceable septum and being attached to a first connection assembly, the first connection assembly comprising:
a first engagement body comprising a first end having a first opening, a second end having a second opening, a first cavity between the first and second openings, and a first engagement surface at the second end of the first engagement body; and
a first flexible barrier arranged to seal the second opening;
wherein the first end is attached to the container such that the first end forms a seal with the septum.

The container may comprise the pierceable septum at a first end, and a piston at a second end. The piston may be moveable towards the septum, to thereby expel a medicament from the container when the septum has been pierced.

In a third aspect there is provided a second connection assembly for creating an aseptic connection with a container (e.g., medicament container), the second connection assembly comprising:
a second engagement body comprising a third end having a third opening, a fourth end having a fourth opening, a second cavity between the third and fourth openings, and a second engagement surface at the fourth end of the second engagement body;
a needle extending through the third opening into the cavity, the needle and the fourth end being axially moveable relative to (e.g., towards) one another; and
a flexible barrier arranged to seal the fourth opening.

As the reader will understand, the second and third aspects may collectively form a connection system according to the first aspect. Moreover, the second and third aspects may respectively be considered as a ‘plug’ and corresponding ‘socket’ (or vice versa).

The first and second flexible barriers may each be semi-permeable. For example, each of the first and second flexible barriers may comprise a breathable sterilizable membrane, such as spunbound polyethylene (e.g., Tyvek (RTM)), polyethersulfone (PES), potytetraftuoreethylene (PTFE), etc. Breathable barriers such as these allow gases, such as steam and water vapor, to travel therethrough, while preventing liquids from passing therethrough. Thus, such breathable barriers provide a barrier to microbes and certain particulates. Alternatively, a non-permeable barrier such as a multilayer polyethylene film or an aluminium foil based film can be utilized.

The connection system may be configured such that the first, second, third and fourth openings are substantially aligned along the same axis, when the first sealing interface forms a seal with the second sealing interface.

The first flexible barrier may be further arranged to cover the first sealing interface. The second flexible barrier may be further arranged to cover the second sealing interface. Thus, sterility of the first and/or second sealing interface(s) is also maintained.

The first sealing interface may comprise a first circumferential seal that surrounds the second opening. For example, the first sealing interface may comprise a first O-ring. Similarly, the second sealing interface may comprise a second circumferential seal that surrounds the fourth opening. For example, the second sealing interface may comprise a second O-ring. A diameter of the first O-ring may substantially match a diameter of the second O-ring.

The first circumferential seal (e.g., first O-ring) may be provided within a first circumferential groove in the first engagement surface. The second circumferential seal (e.g., second O-ring) may be provided within a second circumferential groove in the second engagement surface.

The needle may comprise a free end, wherein the free end is translatable relative to (e.g., towards) the second engagement surface (e.g., from a position in which the free end of the needle is positioned within the second cavity between the third and fourth openings, or alternatively external to the second cavity). Thereby, the free end of the needle may be able to pierce a septum attached to the first engagement body.

In one example, the second engagement surface may be translatable towards the free end of the needle and towards the third opening. Therefore, as the second engagement surface moves towards the third opening, the needle will pass through the fourth opening. Accordingly, when a container having a septum is coupled with the first connection assembly such that the septum forms a seal with the first opening, the needle can extend into the first cavity and pierce the septum—thus establishing a fluid connection with the container.

The needle may be mounted to a needle holder. For example, the needle holder may connect the needle to the third opening and form a seal between the needle and the third opening. The needle holder may be connected to each of the needle and the third opening and may form a seal with each of the needle and the third opening.

The needle may be slidably coupled with the needle holder, such that the needle is translatable relative to the third opening while maintaining its seal. Accordingly, the needle may be moveable through the fourth opening. Accordingly, when a container having a septum is coupled with the first connection assembly such that the septum forms a seal with the first opening, the needle can extend into the first cavity and pierce the septum—thus establishing a fluid connection with the container.

The needle may further be connected to a fluid conduit. That is to say, the end of the needle that is distal from the free end may be connected to a fluid conduit. The fluid conduit may further be connected to a hypodermic needle (as distinct from the needle of the first aspect, which will sometimes be referred to herein as a transfer needle, when necessary to distinguish from the hypodermic needle). Thus, the (transfer) needle may be fluidly connected to the hypodermic needle via the fluid conduit.

The connection system may comprise a first clip mechanism configured to secure the first engagement body to the second engagement body such that the first sealing interface forms a seal with a second sealing interface. The first clip mechanism may comprise at least one indentation on one of the first and second engagement bodies, and a corresponding at least one latch arm on the other of the first and second engagement bodies, wherein the at least one latch arm is configured to engage the at least one indentation so as to maintain the seal between the first and second sealing interfaces. The clip mechanism may be configured to secure the first engagement body to the second engagement body by a snap-fit.

The connection system may comprise a second clip mechanism configured to maintain the connection system in a configuration in which the needle passes through the fourth opening (e.g., so as to pierce a septum connected to the first connection assembly). The second clip mechanism may comprise at least one ridge on one of the first and second engagement bodies, and a corresponding at least one lip on the other of the first and second engagement bodies, wherein the at least one lip is configured to engage the at least one ridge so as to maintain the connection system in the configuration in which the needle passes through the fourth opening. The second clip mechanism may be configured to maintain the connection system in a configuration in which the needle passes through the fourth opening by a snap-fit.

The second engagement body may comprise a first portion at the first end thereof, and a second portion at the second end thereof. The first portion may comprise the third opening, and the second portion may comprise the fourth opening. The first portion may be configured to slide relative to the second portion. The first and second portions may be made from a substantially rigid material, for example a plastically deformable material. The first portion may comprise a circumferential wall which defines a circumferential edge of the second cavity. A circumferential lip may be located at an end of the circumferential wall that is distal from the third opening. The second portion may comprise at least one ridge that is configured to engage the circumferential lip. For example, the second portion may comprise a pair of ridges that are configured to engage the circumferential lip. The second portion may be configured to fit within the circumferential wall of the first portion, such that the circumferential lip engages the at least one ridge as the first portion is made to slide relative to the second portion. The needle is connected to the first portion. For example, the needle may be connected to the first portion so as to be fixed in place relative to the first portion. The second portion may be slidable towards the first end of the second engagement body. The first portion of the second engagement body may comprise a clip mechanism (e.g., the second clip mechanism described above) configured to lock the first portion in place once it reaches the second end of the second engagement body. The needle may extend through the second opening and into the first cavity when the second clip mechanism locks the first and second portions to each other. The clip mechanism may comprise a snap-fit clip mechanism. The clip mechanism may comprise the circumferential lip and the at least one ridge. For example, once the needle has extended through the second opening and into the first cavity, the circumferential lip may lock in place behind a distal end of the at least one ridge, thereby locking the first and second portions to each other so as to prevent their separation.

At least one of first flexible barrier and the second flexible barrier may comprise:
a sealing portion attached to the first engagement surface and covering the second opening; and
a handle portion by which the first flexible barrier is removable from the first connection assembly.

The handle may facilitate easy removal of the at least one of the first flexible barrier and the second flexible barrier from the corresponding opening.

The at least one of the first flexible barrier and the second flexible barrier may further comprise a fold line between the sealing portion and the handle portion, such that the handle portion extends adjacent and substantially parallel to the sealing portion. That is to say, the handle portion may double back on the sealing portion about the fold line. This may further facilitate easy removal of the at least one of the first flexible barrier and the second flexible barrier, by peeling the sealing portion away from the corresponding opening as the handle portion is pulled by a user.

The first and second flexible barriers may be sealed to their respective engagement surfaces in such a way that they are easily removeable, e.g., easily peeled away from their respective engagement surfaces. For example, the first and second flexible barriers may be heat sealed to their respective engagement surfaces. Alternatively, they may be ultrasonically welded to their respective engagement surfaces. They may also be radio frequency sealed or dielectrically sealed. In another example, they may be adhesively coupled to their respective engagement surfaces.

The first connection assembly may be configured to engage an injection end of a container, wherein the injection end of the container comprises a septum arranged to seal the container. That is to say, the first end of the first engagement body may comprise a collar configured to connect to the injection end of the container. The collar may be configured to engage the injection end of the container such that the septum at the connection end of the container forms a seal with the first end of the first engagement body. The container may comprise a vial or cartridge, for example a vial or cartridge containing a medicament.

In a fourth aspect there is provided a container (e.g., medicament container) filling system comprising:
a container;
a bulk storage container for filling the container with a medicament, the bulk storage container having an interior volume that is larger than that of the container; and
a connection system according to the first aspect;
wherein the bulk storage container is attached to one of the first and second connection assemblies, and the container is connected to the other of the first and second connection assemblies.

By using the connection system according to the first aspect to fill the container from the bulk storage container, sterility of the container and of the medicament is not compromised during filling of the container.

The container may comprise a vial, cartridge, or syringe. The bulk storage container may comprise a flexible bag, stainless steel container, etc.

In a fifth aspect there is provided an injection device comprising:
a housing;
a second connection assembly according to the third aspect; and
a further needle (e.g., hypodermic needle) in fluid communication with the needle (e.g., transfer needle) of the second connection assembly, the further needle configured for injecting medicament into an injection site.

The hypodermic needle may be fluidly connected to the transfer needle by a fluid conduit. The hypodermic needle may be oriented non-parallel with the transfer needle. For example, the hypodermic needle may be oriented generally perpendicular to the transfer needle.

The housing may define a skin contact surface. The hypodermic needle may be configured to protrude from the skin contact surface. For example, the hypodermic needle may be translatable between a retracted position in which the hypodermic needle is concealed within the housing, and an injection position in which the hypodermic needle protrudes through an opening in the skin contact surface of the housing. The injection device may comprise an adhesive layer applied to the skin contact surface, wherein the skin contact surface is configured to attach the injection device to the injection site.

The housing may further comprise an enclosure for receiving a container. The enclosure may be in a surface of the housing that is separate from the skin contact surface.

A piston rod may be arranged to engage a piston disposed within an internal volume of a container, the piston rod configured to advance towards the transfer needle of the connection assembly. That is to say, the injection device may be configured to receive a container having a septum and a piston, such that the transfer needle pierces the septum. Following piercing of the septum by the transfer needle, the piston rod may then be configured to engage the piston to thereby expel a medicament from the container via the transfer needle, as the piston rod moves towards the transfer needle. The injection device may further comprise a telescopic screw assembly configured to advance the piston rod towards the needle of the connection assembly.

In a sixth aspect there is provided a method of forming an aseptic fluid pathway using the system of the first aspect, the method comprising:
coupling the first connection assembly with the second connection assembly such that the first sealing interface forms a seal with the second sealing interface;
removing the first and second flexible barriers; and
moving the needle into the first cavity.

Where the first connection assembly of the first aspect is coupled to a container comprising a septum (such that a first end of the first engagement body forms a seal with the septum), the method may comprise moving the needle into the first cavity and through the septum, to thereby establish fluid communication with the container.

In a seventh aspect there is provided a method of forming an aseptic fluid pathway between a container (e.g., medicament container) and an injection device, the method comprising:
coupling the container with the injection device, such that a first sealing interface of the container engages a second sealing interface of the injection device;
removing a first flexible barrier from the first sealing interface, and removing a second flexible barrier from the second sealing interface, to thereby establish an aseptic environment between a needle of the injection device and a septum which seals the container; and
advancing the needle of the injection device towards the septum, such that the needle pierces the septum to thereby form a fluid pathway between the container and the injection device.

The container may be a container according to the second aspect. The injection device may be an injection device according to the fifth aspect.

In an eighth aspect there is provided a method of forming an aseptic environment, the method comprising:
providing a container containing a medicament, the medicament being sealed within the container by a septum;
attaching a connection assembly to the container, the connection assembly comprising a first end having a first opening, a second end having a second opening, and a cavity between the first and second openings, wherein a flexible barrier seals the second opening; and
sterilising the cavity to thereby form an aseptic environment around the septum.

The container may be a container according to the second aspect. Sterilising the cavity may comprise treating the assembled container and connection assembly with sterilising radiation, such as gamma radiation. Alternatively, sterilisation may be performed by an autoclave steriliser. Attaching the connection assembly to the container may comprise attaching the first end of the connection assembly to the container such that the first end of the connection assembly forms a seal with the septum.

According to another aspect of this disclosure, a connection system for use with an injection device includes a first connection assembly, a second connection assembly, and a coupling assembly configured to secure the first and second connection assemblies together. The first includes a first body configured to connect to a medicament container, the first body defining a first opening therethrough configured to be in fluid communication with the medicament container; a first coupling surface on the first body; a first seal adjacent to the first coupling surface; and a first flexible barrier disposed on the first coupling surface, such that the first opening is closed by the first flexible barrier. The second connection assembly includes a second body configured to receive a needle therein, the second body defining a second opening therethrough; a second coupling surface on the second body, the second coupling surface being configured to be placed adjacent to the first coupling surface; a second flexible barrier disposed on the second coupling surface, such that the second opening is closed by the second flexible barrier; and a second seal adjacent to the second coupling surface. When the first and second connection assemblies are secured together, an insertion axis is defined parallel to the needle and extending through the first and second openings of the first and second connection assemblies. When the first and second connection assemblies are secured together, the first and second flexible barriers are configured to be removed, such that the first and second openings fluidly communicate with each other. The needle is configured to be moved through the second opening and the first opening and into the medicament container, such that the needle is in fluid communication with the medicament container. An aseptic connection is formed between the first and second connection assemblies.

Optionally, the needle can be configured to pierce a septum of the medicament container so as to create the fluid communication between the needle and the medicament container.

At least one of the first and second connection assemblies can include a first seal adjacent to the respective first and second coupling surfaces, such that when the first and second connection assemblies are coupled together, the first seal contacts the other of the first and second connection assemblies to form a liquid-tight seal between the first and second connection assemblies.

The first connection assembly can include the first seal, and the second connection assembly includes a second seal, and wherein the first and second seals are configured to contact each other when the first and second connection assemblies are coupled together.

The coupling assembly can include a protrusion on one of the first and second bodies and a receptacle on the other of the first and second bodies, the receptacle being configured to receive the protrusion therein when the first and second connection assemblies are moved towards each other. When the protrusion is in the receptacle, movement of the first and second connection assemblies away from each other can be precluded.

The needle can be configured to be movable between a first position, a second position, and a third position, the first, second, and third positions being axially displaced from one another along the insertion axis. When the needle is in the first position, a tip of the needle can be entirely within the second connection assembly and can be disposed so as not to extend out of the second opening. When the needle is in the second position, the tip of the needle can be disposed within the first opening of the first connection assembly. When the needle is in the third position, the tip of the needle can be configured to be in the medicament container.

The needle can be disposed on a needle hub movable within a chamber defined by the second body of the second connection assembly. The chamber can have a first segment having a first diameter and a second segment having a second diameter different from the first diameter. The friction between the needle hub and the first segment can be greater than the friction between the needle hub and the second segment. The needle hub can include a keyed protrusion thereon configured to be received into a corresponding keyed receptacle on an injection device.

According to another aspect of this disclosure, an injection device for providing a medicament to a user can include a housing having an injection needle configured to be moved to an injection site; a medicament container disposed in the housing, the medicament container being configured to receive the medicament therein; a transfer needle configured to be placed into fluid communication with the injection needle and the medicament container, such that the medicament can be moved from the medicament container to the injection needle through the transfer needle; and a connection system configured to form an aseptic connection between the transfer needle and the medicament container.

Optionally, the connection system can include a first connection assembly and a second connection assembly. The first connection assembly can include a first body configured to connect to the medicament container. The first body can define a first opening therethrough configured to be in fluid communication with the medicament container. The first connection assembly can also include a first coupling surface on the first body; a first seal adjacent to the first coupling surface; and a first flexible barrier disposed on the first coupling surface, such that the first opening is closed by the first flexible barrier. The second connection assembly can include a second body configured to receive the transfer needle therein, the second body defining a second opening therethrough; a second coupling surface on the second body, the second coupling surface being configured to be placed adjacent to the first coupling surface; a second flexible barrier disposed on the second coupling surface, such that the second opening is closed by the second flexible barrier; and a second seal adjacent to the second coupling surface. When the first and second connection assemblies are secured together, an insertion axis can be defined parallel to the transfer needle and extending through the first and second openings of the first and second connection assemblies. When the first and second connection assemblies are secured together, the first and second flexible barriers can be configured to be removed, such that the first and second openings fluidly communicate with each other. The transfer needle can be configured to be moved through the second opening and the first opening and into the medicament container, such that the transfer needle is in fluid communication with the medicament container.

The transfer needle can optionally be disposed on a needle hub movable within a chamber defined by the second body of the second connection assembly. The needle hub can include a keyed protrusion thereon configured to be received into a corresponding keyed receptacle on the housing.

Optionally, the housing can define a receptacle configured to receive the medicament container and the connection system therein. The connection system can be configured to have a first rotational orientation and a second rotational orientation angularly displaced from the first rotational orientation. The housing can include a first keyed feature extending into the receptacle, the receptacle being configured to preclude movement of the connection system into the receptacle when the connection system is in the first orientation, and to allow movement of the connection system into the receptacle when the connection system is in the second orientation.

The connection system can optionally include a coupling assembly configured to secure the first and second connection assemblies together.

The medicament container can optionally include a septum thereon that provides a liquid seal between the medicament in the medicament container and the connection system. The septum can be configured to be pierced by the transfer needle such that the fluid communication is formed between the transfer needle and the medicament container.

The housing can include a movable door configured to be opened and closed, the medicament container being receivable onto the door.

Optionally, the injection device can include an activation button assembly configured to cause movement of the injection needle from a first position, in which the injection needle is spaced from the injection site, to a second position, in which the needle contacts the injection site.

According to another aspect of this disclosure, a method of forming an aseptic connection between a medicament container and an injection needle in an injection device is described. The method can include the step of introducing the medicament container into a receptacle of a housing of the injection device. The medicament container can have a first end defining a container opening through which a medicament is movable into or out of the medicament container. The method can include the step of engaging a first connection assembly of a connection system with the first end of the medicament container, the first connection assembly having a first body and a first opening extending through the first body, the first opening of the first connection assembly being positionable so as to be in fluid communication with the container opening. The method can further include the step of engaging the first connection assembly with a second connection assembly of the connection system, the second connection assembly having a second body and a second opening extending through the second body, the second opening being positionable to be in fluid communication with the first opening. The method can further include the step of actuating a transfer needle disposed in the second connection assembly to be moved into the container opening such that a fluid communication is formed between the medicament container and the transfer needle.

Optionally, the first end of the medicament container can include a septum configured to seal the container opening, and the step of actuating the transfer needle can include piercing the septum with the transfer needle.

The transfer needle can be disposed on a needle hub slidably movable within the second body of the second connection assembly, and the step of actuating the transfer needle can include causing a relative slidable translation between the needle hub and the second body. The method may optionally further include moving the second connection assembly toward a wall of the housing of the injection device and contacting the wall with the needle hub, such that the wall prevents movement of the needle hub but permits slidable movement of the second body relative to the needle hub, such that the transfer needle is moved toward the first connection assembly.

Optionally, at least one of the first and second connection assemblies can include a sealing element, the method further comprising contacting the sealing element with the other of the first and second connection assemblies to form a liquid-tight seal between the first and second connection assemblies. Optionally, the sealing element can include an O-ring or a gasket. The sealing element can include a rubber, a polymer, or both.

Optionally, at least a first flexible barrier can be disposed on one of the first and second connection assemblies between the first opening of the first connection assembly and the second opening of the second connection assembly. The method can further include the step of removing the at least first flexible barrier after the first and second connection assemblies are engaged. Optionally, the first connection assembly can include the first flexible barrier, and the second connection assembly can include a second flexible barrier. The method can include steps of removing the first and second flexible barriers after the first and second connection assemblies are engaged.

The method can optionally include a step of securing the first connection assembly to the second connection assembly by engaging a first protrusion on one of the first and second connection assemblies with a corresponding first receptacle on the other of the first and second connection assemblies. The first connection assembly can be precluded from being separated from the second connection assembly when the first protrusion is engaged with the second protrusion.

Optionally, the housing can include a protrusion extending into the receptacle of the housing, the protrusion being configured to prevent translation of the connection system within the receptacle of the housing when the connection system is in a first insertion orientation, and the protrusion being configured to permit translation of the connection system within the receptacle when the connection system is in a second insertion orientation. The method can optionally include a step of rotating the connection system by a predetermined distance to transition the connection system from the first insertion orientation to the second insertion orientation. The method can include rotating the connection system by 15 to 180 degrees to transition the connection system from the first insertion orientation to the second insertion orientation.

Optionally, a door can be disposed on the housing and be movable relative to the housing. The door can define at least a portion of the receptacle of the housing. The method may optionally include moving the door to an open position to permit introduction of the medicament container into the receptacle and moving the door to a closed position to preclude removal of the medicament container from the receptacle.

### BRIEF DESCRIPTION OF THE DRAWINGS

The invention will be described in more detail with reference to a number of non-limiting examples as shown in the following drawings, in which:

FIG. 1 shows a schematic illustration of an injection device;

FIG. 2 shows an example injection system corresponding to FIG. 1;

FIG. 3 shows a cutaway view of the injection system of FIG. 2;

FIG. 4a shows a connection system according to the present disclosure, in an unconnected state;

FIG. 4b shows the connection system of FIG. 4a in a first connected state;

FIG. 4c shows the connection system of FIG. 4a in a second connected state, with a sterile connection established;

FIG. 4d shows the connection system of FIG. 4a in a third connected state, with a fluid path established;

FIG. 4e shows a component of the connection system of FIGS. 4a-4d;

FIG. 4f shows another component of the connection system of FIGS. 4a-4d;

FIG. 4g shows a cross-sectional view of another component of the connection system of FIGS. 4a-4d;

FIG. 5a shows a first connection assembly according to a further example of the present disclosure;

FIG. 5b shows a second connection assembly according to a further example of the present disclosure;

FIG. 6 shows the first and second connection assemblies of FIGS. 5a and 5b as the seal is removed;

FIG. 7 is a schematic illustration showing a system for filling a medicament container according to an example of the present disclosure;

FIG. 8 is a flow chart showing a method of forming an aseptic fluid pathway according to the present disclosure;

FIG. 9 shows a perspective view of a connection system according to another aspect of the present disclosure;

FIG. 10 shows an exploded cross-sectional view of a portion of the connection system of FIG. 9;

FIG. 11a shows a cross-sectional view of the connection system of FIG. 9 in a first connection state;

FIG. 11b shows a cross-sectional view of the connection system of FIG. 9 in a second connection state;

FIG. 11c shows a cross-sectional view of the connection system of FIG. 9 in a third connection state;

FIG. 12a shows a cross-sectional view of a portion of an injector device in a first connection state according to an aspect of this disclosure;

FIG. 12b shows a cross-sectional view of the portion of the injector device of FIG. 12a in a second connection state;

FIG. 12c shows a cross-sectional view of the portion of the injector device of FIG. 12a in a third connection state; and

FIG. 13 depicts a flow chart of an exemplary method of use of an injection device.

Like reference numerals are used for like components throughout the drawings.

### DETAILED DESCRIPTION OF ILLUSTRATIVE EMBODIMENTS

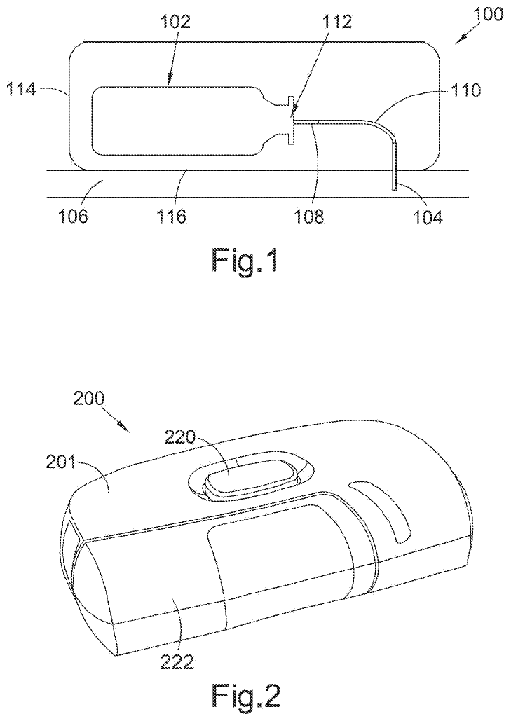

FIG. 1 shows a schematic of an injection system comprising an on-body injection device 100 and a medicament container 102. The injection system is shown in the assembled state in this schematic, in which the medicament container 102 is coupled with the injection device 100, and the injection device 100 is ready for performing an injection.

As shown in the schematic of FIG. 1, the injection device 100 includes a hypodermic needle 104 for piercing the skin 106 of a patient. A transfer needle 108 is fluidly connected to the hypodermic needle 104 and can define a fluid conduit 110 between the transfer needle 108 and the hypodermic needle 104. The medicament container 102 may include a septum 112 that is configured to be pierced by a free end of the transfer needle 108, as will be described in further detail below. This arrangement can allow for the hypodermic needle 104 to be in fluid communication with the medicament container 102 via the transfer needle 108 and the fluid conduit 110, such that a medicament from the medicament container 102 can be dispensed through the hypodermic needle 104.

The schematic showing the injection device 100 depicts a housing 114, which is configured to receive therein the components of the injection device 100, such as the medicament container 102. The injection device 100 can include a skin contact surface 116 configured to contact the skin 106 of the patient. An adhesive layer may be disposed on, or adjacent to, the skin contact surface 116 to improve attachment to the skin 106.

In some embodiments, it is contemplated that the hypodermic needle 104 can comprise part of an infusion set (not shown) spaced from the housing 114, rather than being directly attached to the housing 114. In such devices, the injection device 100 can pump the medicament to the remotely located hypodermic needle 104 for delivery to a patient.

It should be appreciated that FIG. 1 depicts a non-limiting schematic of an injection device 100 and may not include all features of the injection device 100 for simplicity. Components described above with respect to FIG. 1 can be similar to, or substantially the same as, similarly named components described elsewhere in this application with respect to further embodiments.

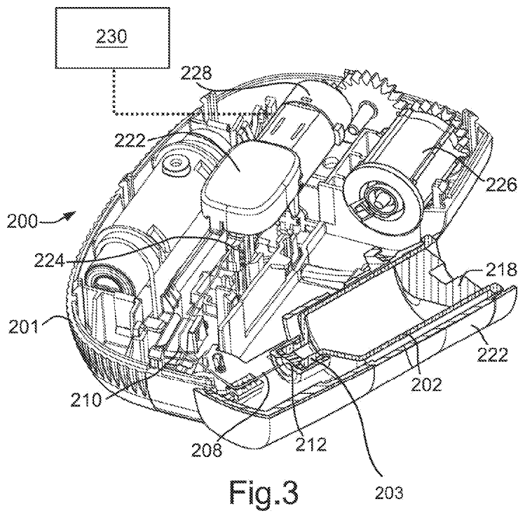

FIGS. 2 and 3 show an example wearable injection device 200 in which a connection system according to the present disclosure can be implemented in more detail. FIG. 2 shows the injection device 200 in an assembled state ready for attachment to an injection site (e.g., the skin 106). As the reader will understand, the injection device 200 may be similar to the injection device 100 described above and depicted in FIG. 1, and descriptions of the components listed above can apply to the embodiments of FIGS. 2 and 3 as well. The injection device 200 can include a housing 201 and an actuator 220. The actuator 220 may be configured to cause movement of a hypodermic needle, as will be described further below.

The injection device 200 includes a hypodermic needle configured to be disposed at the desired injection site, such as in or on the user's skin 106. The hypodermic needle of the injection device 200 may be similar to, or substantially the same as, the needle 104 depicted in the schematic of FIG. 1. The hypodermic needle can be translatable relative to the housing 201 between a retracted position, in which the hypodermic needle is concealed within the housing 201, and an injection position, in which the hypodermic needle protrudes through an opening in the skin contact surface of the housing (see, e.g., the needle 104 shown in the schematic of FIG. 1). Because the hypodermic needle is concealed within the housing 201 in the retracted state, the hypodermic needle does not present an injury hazard to the patient or to another use of the injection device 200. The injection device 200 may be configured such that the hypodermic needle only moves into the injection position once the injection device 200 has been secured to the patient's skin 106.

An actuator 220 may be provided on the housing 201 to initiate an injection procedure. The actuator 220 may include a button, a switch, a trigger, or the like configured to cause the hypodermic needle to be moved from the retracted position to the injection position. The actuator 220 can include a mechanical actuator for advancing the hypodermic needle toward and/or into the injection site (e.g., at the skin 106). Alternatively, the actuator 220 may be configured to initiate an injection procedure under the control of a control unit.

The injection device 200 is configured to receive a medicament container 202 therein through an opening, a hatch, a cutout, a door, or the like. The injection device 200 may include a door 222 configured to selectively allow insertion of the medicament container 202. The medicament container 202 may be introduced into, or onto, the door 222. The door 222 is shown in the closed position in FIG. 2 and in the open position in FIG. 3. It will be appreciated that the injection device 200 and the medicament container 202 of the injection device 200 may be manufactured and/or sold separately, and may be coupled to one another by a user or healthcare professional before use.

Turning now to FIG. 3, the door 222 is shown in the open position, in which the medicament container 202 may be received into the injection device 200. The door 222 comprises or allows access to an enclosure within the housing 201 configured to receive the medicament container 202. The enclosure is accessible when the door 222 is in the open position. Access to the enclosure is precluded when the door 222 is in the closed position. In the closed position, the door 222 may help secure the medicament container 202 within the enclosure of the housing 201. Therefore, in some aspects, the medicament container 202 can only be inserted into the enclosure of the injection device 200 when the door is in the open position.

As can also be seen from FIG. 3, the medicament container 202 can include a septum 212 at a first end and a plunger 218 at a second end opposite the first end. A medicament (not shown) may be received within an internal volume of the medicament container 202. The internal volume is defined by an interior surface of the medicament container 202, the septum 212, and the plunger 218. The septum 212 may prevent the medicament from being moving or being moved from the medicament container 202 at the first end thereon, and the plunger 218 may prevent the medicament from moving or being moved from the medicament container 202 at the second end. The plunger 218 is moveable through the medicament container 202 along an axis extending between the first end and the second end of the medicament container. The plunger 218 may be configured to move in a direction along the axis towards the first end of the medicament container 202. The plunger 218 forms a seal with the interior surface of the medicament container 202 to define the internal volume of the medicament container 202. Movement of the plunger relative to the first end varies the internal volume of the medicament container 202. For example, when the plunger is moved toward the first end, the internal volume is decreased; when the plunger is moved toward the second end, the internal volume is increased. When the medicament is in the internal volume, and the plunger 218 is moved towards the first end, and the internal volume is reduced, pressure is applied to the medicament toward the first end. The applied pressure can result in the medicament being expelled from the medicament container 202 at the first end, for example, through the septum 212. It will be appreciated that the septum 212 can include a desired material configured to withstand a predetermined amount of pressure without rupturing, breaking, or otherwise degrading or allowing the medicament to travel through or around the septum 212. To allow the medicament to exit the medicament container 202 at the septum 212, the septum 212 may be removed, punctured, or pierced to define one or more apertures through which the medicament is permitted to travel.

The injection device 200 may include a transfer needle 208 configured to pierce the septum 212 at the first end of the medicament container 202. The transfer needle 208 may be substantially similar to, or the same as, the transfer needle 108 described above. The transfer needle 208 is configured to pierce the septum 212. Upon piercing the septum 212, a fluid flow path may be established between the internal volume of the medicament container 202 and the transfer needle 208. The transfer needle 208 may be hollow and may be configured to receive therein the medicament from within the medicament container 202. As the plunger 218 is moved toward the first end, the medicament may be discharged from the internal volume of the medicament container 202, through the septum 212, and into the transfer needle 208.

As shown in FIG. 3, the injection device 200 further includes an insertion mechanism 224 configured to advance a hypodermic needle (not shown in FIG. 3) from a pre-injection position within the housing to an injection-ready position. A fluid conduit 210 can extend between the transfer needle 208 and the injection needle.

The plunger 218 is advanced along the body of the medicament container 202 by a drive assembly. The drive assembly of the device 200 shown in FIGS. 2 and 3 may include a telescopic screw assembly (TSA) 226 and a motor 228. The TSA 226 is configured to advance a piston rod (e.g., toward the plunger 218). The motor 228 is configured to cause actuation of the TSA 226 to thereby advance the piston rod. A control unit 230 (shown schematically in FIG. 3) is provided to control actuation of the motor 228. The TSA 226 is configured to engage the plunger 218 (e.g., via the piston rod of the TSA 226) to thereby advance the plunger 218 through the medicament container 202.

In the configuration shown in FIG. 3, it can be seen that fluid communication between the transfer needle 208 and the internal volume of the medicament container 202 has not yet been made, and the septum 212 (or other suitable seal) has not yet been pierced by the transfer needle 208. However, before an injection can take place via the injection needle 104, the transfer needle 208 must be brought into fluid communication with the internal volume of the medicament container 202. Sterility of the fluid conduit 210, the transfer needle 208, and the septum 212 should be maintained throughout this process.

As the reader will understand, before assembly of an injection device, there is a risk that a septum or transfer needle exposed to an external environment could become contaminated, compromising the sterility of the connection. This may occur when a transfer needle pierces the septum, and contamination from the needle or the septum may enter the medicament container, thus contaminating the medicament stored within.

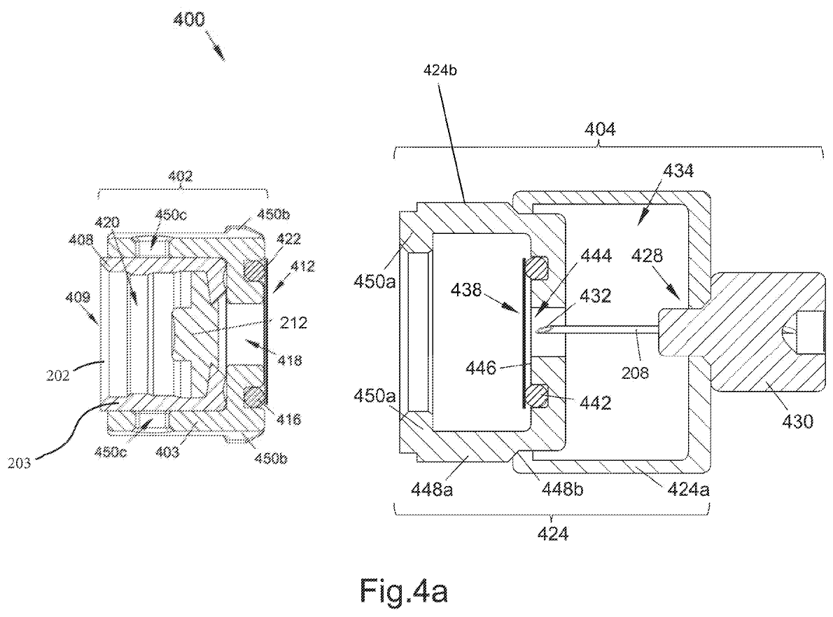

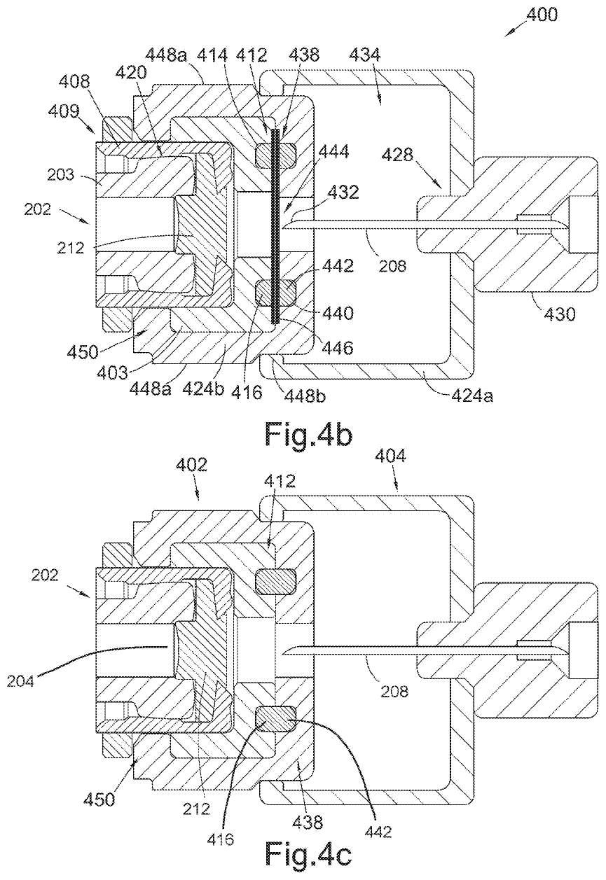

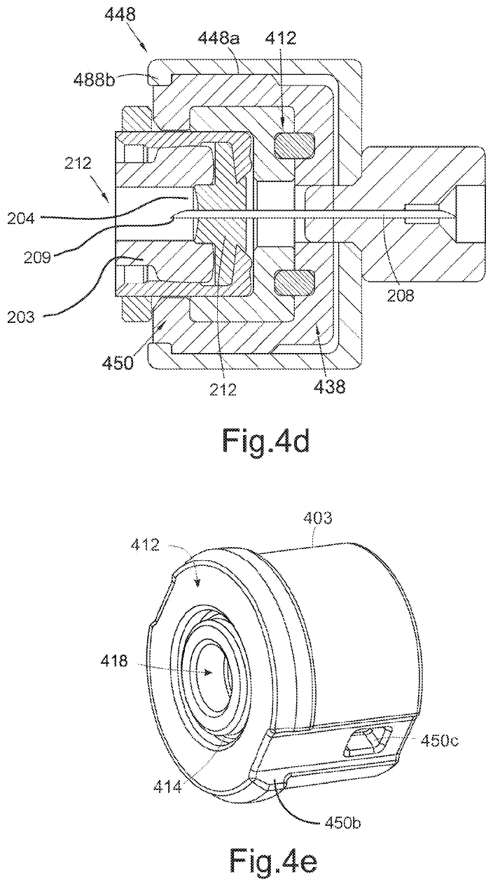

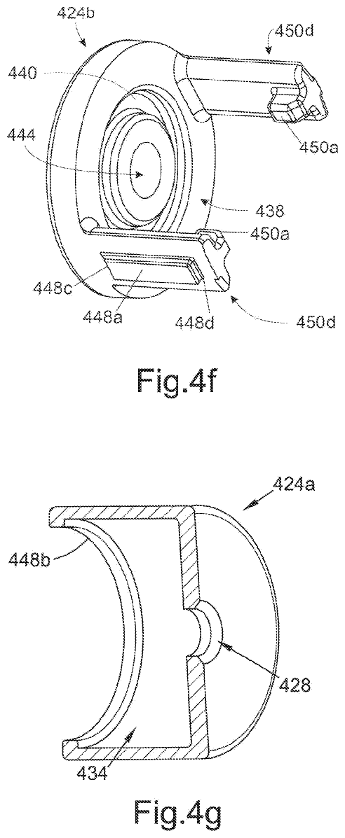

Referring to FIGS. 4-12c, connection systems and associated methods for maintaining the sterility of the transfer needle and the septum will now be described. As the reader will understand, the connection system according to the present disclosure can be used with injection systems as described in FIGS. 1-3, though the present disclosure is not intended to be limited to such. Moreover, when the connection systems according to the present disclosure are used with an injection system of the type described in FIG. 1, a sterile connection between the injection device 100 and the medicament container 102 can be achieved. Similarly, when the connection systems described throughout this application are used with an injection system of the type depicted in FIGS. 2 and 3, a sterile connection between the injection device 200 and the medicament container 202 can be achieved. As the reader will also understand, the connection assemblies and systems described below can be coupled to the septum end of a medicament container or, alternatively, may be disposed within on or within the door components described above. It will further be appreciated that the systems described below may be used to form other sterile connections within a wearable injection device or may be used to form connections in other drug delivery systems (e.g., pen-type injectors comprising a seal medicament cartridge, infusion pumps, and the like).

FIGS. 4a to 4d show a connection system 400 according to the present disclosure. FIG. 4a shows the connection system in an unconnected state. FIG. 4b shows the connection system 400 in a first connected state. FIG. 4c shows the connection system 400 in a second connected state with a sterile connection established. FIG. 4d shows the connection system 400 in a third connected state with a fluid connection established.

The connection system 400 includes a first connection assembly 402 and a second connection assembly 404. The two connection assemblies 402 and 404 are shown separately in FIG. 4a and are shown connected to one another in FIGS. 4b-4d. As the reader will appreciate, the two connection assemblies 402 and 404 may be manufactured and supplied separately. A medicament container 102/202 may be supplied with the first connection assembly 402 attached thereto. Similarly, an injection device 100/200 incorporating the second connection assembly 404 may be supplied as a separate product. It should be understood that an injection device 100 or 200 can be used with the connection system 400, and a medicament container 102 or 202 can be used with the connection system 400, and that this disclosure is not limited to a particular injection device or medicament container. While the drawings may refer to the medicament container 202 for reference, it should be appreciated that the description can also apply to an embodiment referencing the medicament container 102. Components described with respect to the medicament container 202 can be similar to, or substantially the same as, similarly named or functioning components of the medicament container 102.

The first connection assembly 402 includes a first engagement body 403 and is shown in FIGS. 4a-4d as being attached to a medicament container 202. In particular, the first engagement body 403 comprises a collar 408 configured to engage (e.g., such that the collar 408 is secured around) a neck 203 of the medicament container 202, so as to form a seal around the septum 212 of the medicament container 202. The collar 408 terminates at a first opening 409 at a first end of the first engagement body 403. The second end of the first engagement body 403 includes a first engagement surface 412 for engaging a corresponding second engagement surface 438 of a second connection assembly 404 (to be described below). The first engagement surface 412 includes a circumferential groove 414 configured to receive a seal 416. The seal 416 can include an O-ring, a gasket, or another suitable seal. The seal 416 can form a sealing interface on the first engagement surface 412.

The second end of the first engagement body 403 includes a second opening 418, such that a first cavity 420 is formed between the first and second openings 409 and 418. The first cavity 420 extends from the first opening 409 to the second opening 418. The first and second openings 409 and 418 of the first engagement body 402 are arranged such that the neck 203 and the septum 212 of the medicament container 202 are located within the first cavity 420 when assembled together as shown in FIGS. 4a-4d. A first flexible barrier 422 can be affixed to the first engagement surface 412. The first flexible barrier 422 extends over the second opening 418 and the first sealing interface. By sealing the second opening 418, the first flexible barrier 422 can prevent or decrease contamination of the septum 212.

The second connection assembly 404 includes a second engagement body 424. The second engagement body 424 includes a first portion 424a at a first end of the second engagement body 424, and a second portion 424b at the second end of the second engagement body 424. The first portion 424a can be slidably coupled with the second portion 424b as will be described in more detail below. The first portion 424a of the second engagement body 424 includes a third opening 428, at which a needle holder 430 can be attached. The transfer needle 208 can be inserted into the needle holder 430, such that a free end 432 of the transfer needle 208 is positioned within a second cavity 434 of the second engagement body 424. The second portion 424b of the second engagement body 424 includes the second engagement surface 438. The second engagement surface 438 includes a circumferential groove 440 configured to receive a seal 442. The seal 442 can include an O-ring, a gasket, or another suitable seal. The seal 442 forms a sealing interface on the second engagement surface 438. The second end of the second engagement body 424 defines a fourth opening 444, with the second cavity 434 being defined between the third and fourth openings 428 and 444.

A second flexible barrier 446 can be affixed to the second engagement surface 438. The second flexible barrier 446 extends over the fourth opening 444 and the second sealing interface. By sealing the fourth opening 444, the second flexible barrier 446 can prevent or decrease contamination of the transfer needle 208.

The first and second flexible barriers 422 and 446 can each comprise a semi-permeable aseptic membrane. Alternatively, the first and second flexible barriers 422 and 446 can each comprise a non-permeable aseptic membrane. The first flexible barrier 422 and the second flexible barrier 446 can be attached to their respective engagement surfaces so as to be manually removeable. That is to say, the first flexible barrier 422 can be removeable from the first engagement surface 412 to thereby expose the second opening 418 and the first sealing interface. Similarly, the second flexible barrier 446 can be removeable from the second engagement surface 438 to thereby expose the fourth opening 444 and the second sealing interface.

In some aspects, the first and second flexible barriers 422 and 446 can comprise a single flexible sheet. That is, each of the first and second flexible barriers 422 and 446 can be separate discrete portions of the same unitary component. Accordingly, removal of the singular unitary sheet can cause removal of each of the first and second flexible barriers 422 and 446, either serially or simultaneously. In such an arrangement, the first flexible barrier 422 is connected to the second flexible barrier 446. The first and second flexible barriers 422 and 446 may be separated from each other prior to use, or, alternatively, may remain connected to each other throughout use.

As shown in FIG. 4c, when the first and second flexible barriers 422 and 446 are removed while the first and second engagement surfaces 412 and 438 are in abutment, the seals 416 and 442 directly engage each other and collectively form a fluid seal. Thus, the first and second sealing interfaces continue to prevent contamination of the septum 212 and of the transfer needle 208 even when the first and second flexible barriers 422 and 446 are removed. Therefore, once the first and second connection assemblies 402 and 404 have been attached to one another, the first and second flexible barriers 422 and 446 can be removed to provide access for the transfer needle 208 to pierce the septum 212 without compromising the sterility of either the septum 212 or the transfer needle 208. This arrangement is depicted in FIG. 4c.

In order to ensure sterility of the septum 212 and the transfer needle 208 after the first and second flexible barriers 422 and 446 have been removed, the connection system 400 can include a first clip mechanism 450. The first clip mechanism 450 includes a snap-fit clip mechanism that is configured to maintain engagement of the first and second engagement surfaces 412 and 438. In particular, when the first and second engagement surfaces 412 and 438 are bought into engagement, the first clip mechanism 450 can snap into place, thus preventing their separation. The first clip mechanism 450 (labeled generally in FIGS. 4b-4d) includes a lip 450a (labeled in FIGS. 4a and 4f) on one of the first and second connection assemblies 402 and 404, and a corresponding ridge 450b (labeled in FIGS. 4a and 4e) on the other of the first and second connection assemblies 402 and 404. Each lip 450a can be disposed on a corresponding latch arm 450d (shown in FIG. 4f). Each of the first and second connection assemblies 402 and 404 may include a plurality of lips 450a and/or corresponding ridges 450b. In some aspects, the first connection assembly 402 may include both, a lip 450a and a ridge 450b, and the second connection assembly 404 may similarly include both, a lip 450a and a ridge 450b. As shown in the embodiment of FIGS. 4a-4d, the first connection assembly 402 may include a pair of ridges 450b, and the second connection assembly 404 may include a pair of inward-facing lips 450a configured to contact and engage with the corresponding ridges 450b. During assembly, the each of the inward-facing lips 450a can deflect over their respective corresponding ridge 450b and then move along an outer surface of the first engagement body 403 toward one or more indentations 450c (labeled in FIGS. 4a and 4e) defined on the first connection assembly 402. The indentations 450c can be configured to receive the lips 450a such that, when the lips 450a are within the indentations 450c, the lips 450a are precluded from further movement toward and/or away from the first connection assembly 402. The engagement between the lips 450a and the indentations 450c may include a snap fit, a friction fit, an interference fit, or another suitable mechanical arrangement. The first and second connection assemblies 402 and 404 can be considered connected or engaged with one another when the lips 450a are secured within the indentations 450c. In some aspects, the first clip mechanism 450 may be designed to prevent decoupling of the first and second connection assemblies 402 and 404 after they are coupled. It will be appreciated that the components can be decoupled using excessive force or tools, but such decoupling may not be intended for safety and security related to using the injection devices. In some aspects, the first clip mechanism 450 may be configured to be releasable, such that the first and second connection assemblies 402 and 404 may be decoupled by the user by either applying a predetermined decoupling force, using a tool, and/or manipulating one or more components of the first clip mechanism 450 or the injection device.

When a user removes the first and second flexible barriers 422 and 446 from the first and second engagement surfaces 412 and 438, the first clip mechanism 450 prevents separation of the first and second sealing interfaces, such that sterility of the septum 212 and of the transfer needle 208 is not compromised. The first clip mechanism 450 is illustrated in further detail in FIGS. 4e and 4f.

Although the first clip mechanism 450 described above comprises a pair of inward-facing lips 450a deflecting over a pair of corresponding ridges 450b, the skilled person will appreciate that other structural configurations can be used to achieve a snap-fit engagement as described above. For example, the skilled person will appreciate that instead of a pair of inward-facing lips configured to deflect over a pair of corresponding ridges, the snap fit clip can comprise three or more inward facing lips configured to engage one or more corresponding ridges.

Because the first portion 424a of the second engagement body is slidably coupled with the second portion 424b, the transfer needle 208 is thereby moveable towards the second engagement surface 438 of the second connection assembly 404. In so doing, the transfer needle 208 moves towards the fourth opening 444 and into the configuration shown in FIG. 4d in which the transfer needle 208 passes through the second and fourth openings 418, 444, and into the first cavity 420. Once the transfer needle 208 has pierced the septum 212 as shown in FIG. 4d (thus establishing fluid communication between the medicament container 202 and the transfer needle 208), a second clip mechanism 448 can secure the components in place, thereby preventing separation. The second clip mechanism 448 may include a snap-fit clip mechanism that is configured to maintain engagement between the first and second portions 424a and 424b of the second engagement body 424 in a position in which the needle 208 extends through the septum to form a fluid connection with the medicament container 202.

With continued references to FIGS. 4a-4g, the second clip mechanism 448 can include one or more ridges 448a that extend along an outer surface of the second portion 424b of the second engagement body. The second clip mechanism 448 can include one or more corresponding lips 448b, which protrude inwardly from the first portion 424a of the second engagement body. A plurality of ridges 448a may be disposed radially on the second portion 424b. The first portion 424a may include a plurality of corresponding lips 448b. In some aspects, the first portion 424a may include a single lip 448b extending circumferentially around the first portion 424a and configured to engage with each of the ridges 448a. When the second engagement surface 438 is moved towards the third opening 428, the circumferential lip 448b can be moved over the corresponding ridges 448a until the transfer needle 208 has pierced the septum 212. At this point, the circumferential lip 448b will lock in place behind the distal ends of the ridges 448a, as illustrated in FIG. 4d. Withdrawal of the transfer needle 208 from the septum 212 is thereby prevented.

Although the second clip mechanism 448 described above a circumferential lip 448b configured to move over corresponding ridge 448a before locking in place, the skilled person will appreciate that other snap-fit configurations can be provided to provide the engagement as described above. For example, the skilled person will appreciate that instead of a circumferential lip 448b, the second clip mechanism 448 can comprise two or more inward-facing lips 448a configured to deflect over one or more corresponding ridges 448a.

FIG. 4e shows indentations 450c (one of which is concealed from view in FIG. 4e) of the first clip mechanism 450. FIG. 4e shows the first engagement body 403 of the first connection assembly 402. The indentation 450c is formed in a side surface of the first engagement body 403 and is spaced back from the first engagement surface 412 and corresponding groove 414.

FIG. 4f shows the second engagement portion 424b of the second connection assembly 404. FIG. 4f depicts corresponding latch arms 450d of the first clip mechanism 450, from which the inward-facing lips 450a extend. The latch arms 450d extend from the second engagement surface 438 having the second groove 440.

The latch arms 450d are configured to engage the indentations 450c (via lips 450a), to thereby prevent separation of the first and second connection assemblies 402 and 404 once the first and second engagement surfaces 412 and 438 are in abutment with one another. Additionally, because the latch arms 450d are provided on opposing lateral edges of the second engagement portion 424b with gaps therebetween, a user is able to easily remove the first and second flexible barriers 422 and 446 even when the first and second engagement surfaces 412 and 438 are in abutment. Also shown in FIG. 4f is the ridge 448a as discussed above.

FIG. 4f also shows that each of the ridges 448a is formed on an outer surface of a respective one of the pair of latch arms 450d. Accordingly, they are located on opposite lateral edges of the second engagement portion 424b from one another. The ridges 448a extend in the longitudinal direction, i.e., in a direction that is parallel with the orientation of the transfer needle. Accordingly, they encourage motion in the longitudinal direction, with no axial twisting, when the second engagement surface 438 moves towards the third opening 428.

FIG. 4g shows a cross-section through the first portion 424a of the second engagement body 424. The circumferential lip 448b can clearly be seen from this view. FIG. 4g also shows the second cavity 434 as being located generally between the third opening 428 and the circumferential lip 448b.

The circumferential lip 448b is configured to engage the ridges 448a, to thereby prevent movement of the second engagement surface 438 relative to the third opening 428 when the needle has pierced the septum 212. As a user moves the second engagement surface 438 towards the third opening 428 by pushing the first portion 424a of the second engagement body 424 towards the first engagement body 403, the circumferential lip 448b abuts a near edge 448c of the ridges 448b. Under the force applied by the user, plastic deformation of the connection assembly allows the circumferential lip 448b to move over the ridges 448a. Once the transfer needle 208 has pierced the septum 212, the circumferential lip 448a can then be moved past the far edge 448d of the ridges 448b and snap into place behind the far edge 448d of the ridges 448b. Accordingly, separation of the components is prevented. In particular, because the near edge 448c of each ridge 448b is sloped, whereas the far edge 448d of each ridge 448b forms a right-angle with the outer surface of the outer surface of the first portion 424a, the force required to pierce the septum with the needle is less than the force that would be required to separate the components after they have been coupled. Accordingly, separation of the components once the transfer needle 208 has pierced the septum 212 is substantially prevented. That is to say, it is not possible to separate the components again without causing permanent damage to the connection assembly 400.

Although the connection assembly 400 is depicted with reference to the injection device 100, it will be appreciated that the connection assembly 400 can be utilized with the injection device 200 or with another suitable injection device. If used with the injection device 200, it will be understood that numerical reference to components specific to the injection device 100 can be replaced with, or supplemented with, corresponding like-named or like-functioning components described with respect to the injection device 200 previously.

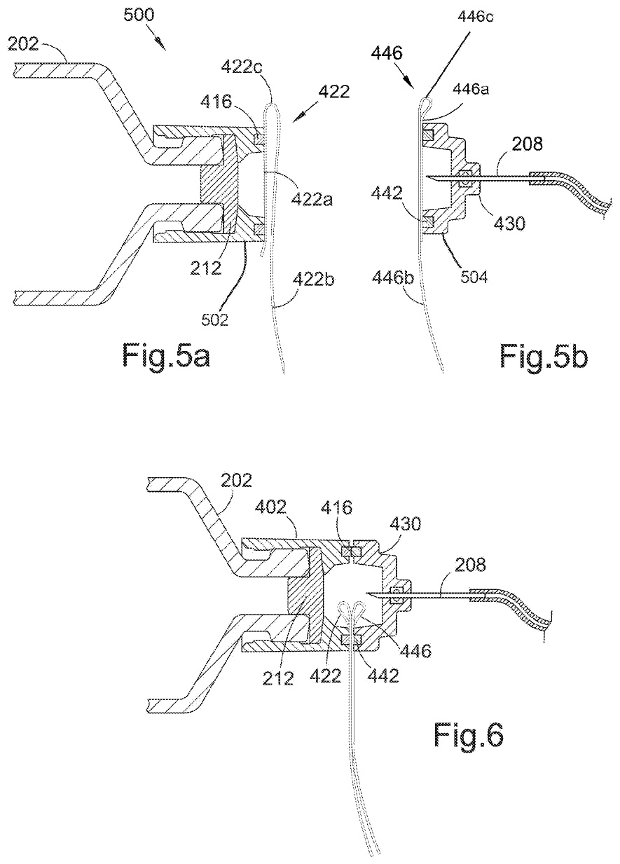

FIGS. 5a-6 depict a connection assembly 500 according to another aspect of this disclosure. FIG. 5a shows a first connection assembly 502, and FIG. 5b shows a second connection assembly 504 configured to be coupled with the first connection assembly 502. FIG. 6 shows the connection assembly 500 in a coupled configuration, where the first connection assembly 502 and the second connection assembly 504 are coupled to each other. For ease of illustration, some features of the first connection assembly 400 from FIGS. 4a-4g have been omitted. However, as the reader will understand, the connection assembly 500 of FIGS. 5a-6 may include any number of the features shown and described with respect to the connection assembly 400 of FIGS. 4a-4g. Specifically, the first connection assembly 502 and the second connection assembly 504 may include some or all of the features described with respect to the first connection assembly 402 and the second connection assembly 404, respectively.

Referring to FIG. 5a, the first flexible barrier 422 can be adhered to a seal 416 on the first connection assembly 502 so as to seal the second opening 418. The adherence can be by any of heat sealing, adhesive coupling, ultrasonic welding, radio frequency sealing, dielectric sealing, or another suitable technique. The first flexible barrier 422 can include a sealing portion 422a, which is adhered to the seal 416, and a handle portion 422b, which is attached to the sealing portion 422a and hangs loose or extends from the housing, such that the user can grasp the handle portion 422b. A user can thus easily peel the first flexible barrier 422 away from the first engagement surface 412 by grasping and pulling on the handle portion 422b. The interface between the sealing portion 422a and the handle portion 422b can include a fold line 422c.

Referring to FIG. 5b, the second connection assembly 504 can include a second flexible barrier 446 that is similar to the first flexible barrier 446 depicted in FIG. 5a. In particular, the second flexible barrier 446 has a sealing portion 446a that is adhered to the seal 442, and a handle portion 446b that is attached to the sealing portion 446a and hangs loose. The interface between the sealing portion 446a and the handle portion 446b can include a fold line 446c.

FIG. 5b also shows an alternative arrangement for translating the transfer needle 208 through the second cavity 434. In particular, the needle 208 can be slidably coupled to the needle holder 430.

FIG. 6 shows how the connection assemblies of FIGS. 5a and 5b may be used together to form respective sterile enclosures for the transfer needle 208 and the septum 212. FIG. 6 further depicts the first and second flexible barriers 446 of the first and second connection assemblies 502 and 504 being partially peeled from their respective components as described above. Peeling the flexible barriers 446 away can form a sterile cavity through which the transfer needle 208 can be advanced to pierce the septum 212.

As shown in FIG. 6, with the barriers 442 and 446 at least partially removed, the first and second sealing interfaces 416 and 416 engage to form an enclosed and aseptic cavity through which the transfer needle 208 can advance to pierce the septum 212. In this way, an aseptic connection may be formed between the transfer needle 208 and the internal volume of the medicament container 202.

It will be appreciated that the connection system 500 of FIGS. 5a-6 can be utilized with the injection devices 100, 200, or another suitable injection device. Although FIGS. 5a-6 refer to components of the injection device 200, this is for reference only and is not intended to limit the applicability of the connection system 500.

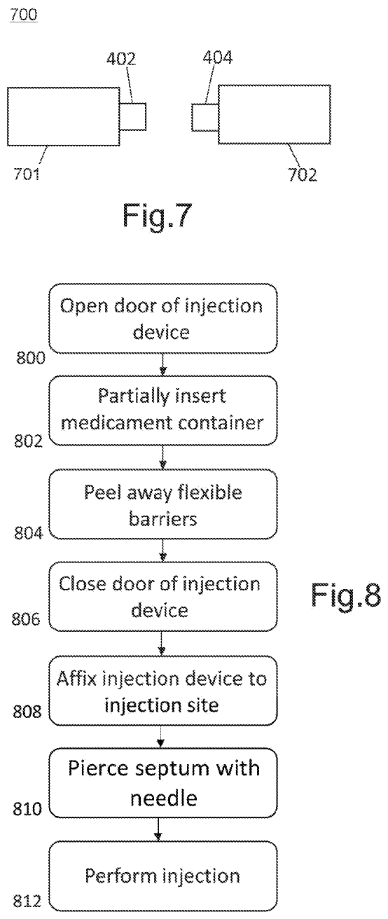

FIG. 7 shows a schematic of a container filling system 700 according to the present disclosure. As shown, the container filling system comprises a first container 701 connected to the first connection assembly 402; and a second container 702 connected to the second connection assembly 404. The first container 701 may be a vial or cartridge, and the second container 702 may be a large container. Alternatively, the first container 701 may be the large container, and the second container 702 may be the vial or cartridge. Accordingly, it is possible to fill the vial or cartridge from the large container without compromising sterility of the vial or the cartridge (or of the medicament itself). The large container may be a flexible bag or may be of rigid construction. Although depicted with the components of the connection system 400, it should be appreciated that the container filling system 700 can be utilized with components of the connection system 500 or with components of another suitable connection system.

FIG. 8 shows a method of forming an aseptic fluid pathway according to the present disclosure. The method of FIG. 6 is performed using the connection system of FIGS. 4a-4c.

At step 800, a user opens a door 122 of an on-body injection device 100 that comprises a second connection assembly 404 according to FIGS. 4a-4c. By opening the door 122, an enclosure in the injection device 100 is revealed. The enclosure contains a second connection assembly 404 according to FIGS. 4a-4c.

At step 802, a user selects a medicament container 102 that contains a correct medicament type and dose for a treatment to be administered, and which comprises a first connection assembly 402 according to FIGS. 4a-4c, with a septum 212 of the medicament container forming a seal with the collar 408 of the first connection assembly. The user couples the injection device 100 with the medicament container 102, by partially inserting the medicament container 102 into the enclosure. In so doing, the first sealing interface of the first connection assembly 402 engages a second sealing interface of the second connection assembly, and the first clip mechanism 450 engages to hold the first and second connection assemblies in place. This corresponds to the first state illustrated in FIG. 4b.

At step 804, the user removes the first and second flexible barriers 422, 446. In particular, the user peels away the first and second flexible barriers, thereby removing the barrier between the transfer needle 108 and the septum 112. This corresponds to the second state illustrated in FIG. 4c.

At step 806, the user closes the door 122 of the injection device 100. In so doing, the medicament container 102 is moved into a position within the injection device 100 in which it is ready for an injection to be performed. In particular, the second end of the medicament container (which comprises the piston 118) aligns with the piston rod of the injection device 100.

At step 808, the user peels away an adhesive covering on the skin contact surface 116 of the injection device 100, and affixes the skin contact surface 116 of the injection device 100 to the patient's skin, ready for an injection to be performed. The adhesive on the skin contact surface 116 retains the injection device 100 in place.

At step 810, the transfer needle 108 is moved through the fourth opening 444 and towards the septum 112, until it pierces the septum 112. Once the septum 112 is pierced, the second clip mechanism 448 engages to hold the transfer needle 108 in place. In this state, a fluid pathway between the medicament container 102 and the injection device 100 has been established. This corresponds to the third state illustrated in FIG. 4d.

At step 812, the user presses the button 120, thereby activating the injection mechanism, such that an injection is performed. The medicament is delivered to the patient through the hypodermic needle 104.

FIG. 8 shows a method of forming an aseptic environment according to an example of the present disclosure. The method is performed with the medicament container 102 from FIG. 1a, and with the first connection assembly 402 from FIGS. 4a-4d.

It should be understood that, although the embodiment of FIG. 1 was referenced in the method 800 described above, the above steps can also apply to other embodiments described herein, such as the connection system 200 and/or the connection system 400.

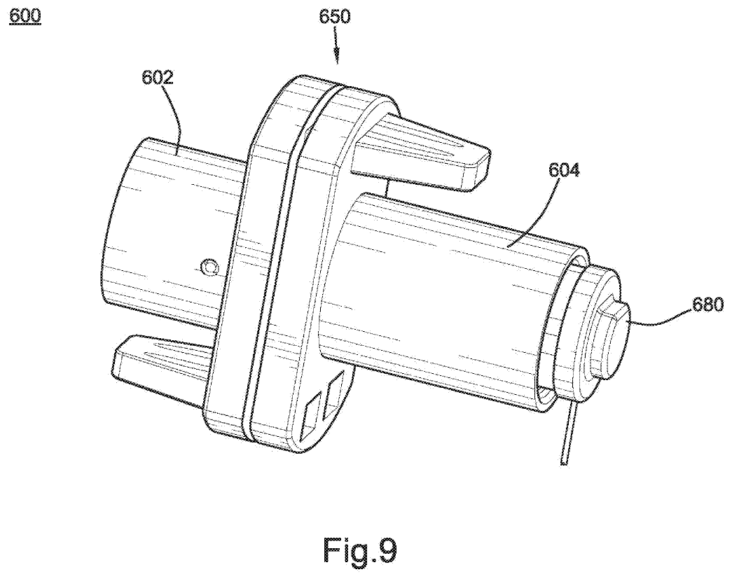

FIGS. 9-12c depict a connection system 600 according to yet another embodiment. The connection system 600 can be utilized with the injection device 100, 200, or another suitable injection device. Components described with respect to the connection system 600 may be similar to, or the same as, similarly named and referenced components described elsewhere in this application. The connection system 600 includes a first connection assembly 602 and a second connection assembly 604. The first and second connection assemblies 602 and 604 can be coupled together. In some aspects, the coupling may be intended to be irreversible, such that the first and second connection assemblies 602 and 604 cannot be readily uncoupled from one another after coupling without applying excessive force, damaging the connection system 600, and/or requiring a specialized tool. The first and second connection assemblies 602 and 604 may be coupled via a coupling assembly 650 having respective components on each of the first and second connection assemblies 602 and 604 as will be described below.

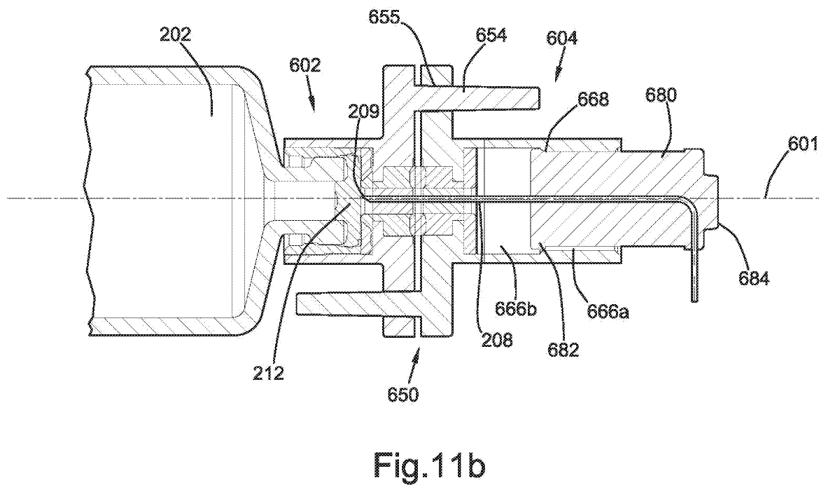

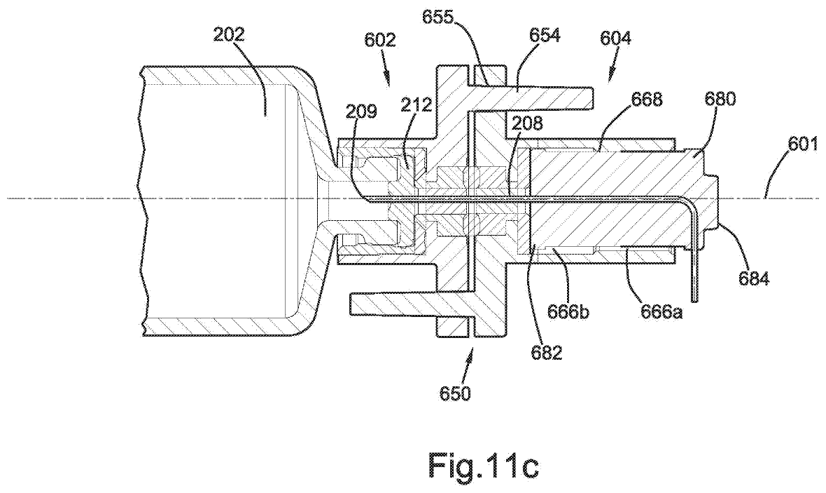

Referring to FIGS. 11a-11c, the first connection assembly 602 may receive thereon or therein, or otherwise couple or attach to, the medicament container 202. The engagement between the first connection assembly 602 and the medicament container 202 may be via any suitable mechanism, such as any of those described elsewhere in this application. For example, a body 603 of the first connection assembly 602 may define a receptacle or another suitable engagement interface configured to connect with a neck 203 of the medicament container 202 such that the septum 212 is suitably secured to, or within, the first connection assembly 602. An opening 204 can be defined on the medicament container 202 at one end thereof, for example, at the neck 203. The interior of the medicament container can be accessible via the opening 204, and medicament can be introduced into, or removed out of, the medicament container 202 through the opening 204. An opening 618 is defined through the body 603 and is configured to receive the transfer needle 208 therein during use. When the medicament container 202 is connected with the first connection assembly 602, the septum 212 can be accessible via the opening 618. The first connection assembly 602 defines a first coupling surface 605 on the body 603 that is configured to be placed adjacent to, or in contact with, a corresponding coupling surface on the second connection assembly 604 as will be described below. The first connection assembly 602 may include a seal 616 thereon. The seal 616 can be on, in, or adjacent to the opening 618. The seal 616 can be on, or adjacent to, the first coupling surface 605. The seal 616 can include an a gasket, or another suitable seal. The seal 616 can form a sealing interface on the first connection assembly 602 and can be contacted with one or more components of the second connection assembly 604 as will be explained below.

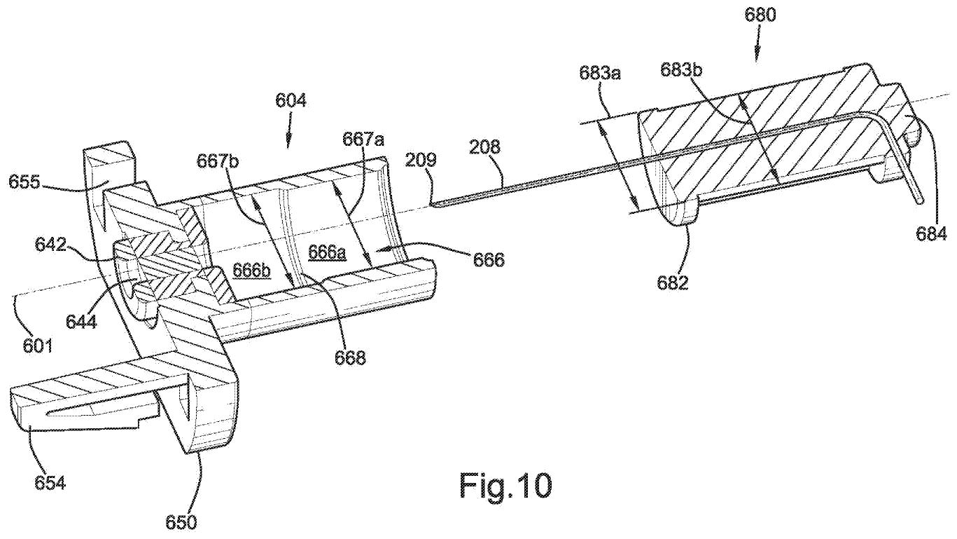

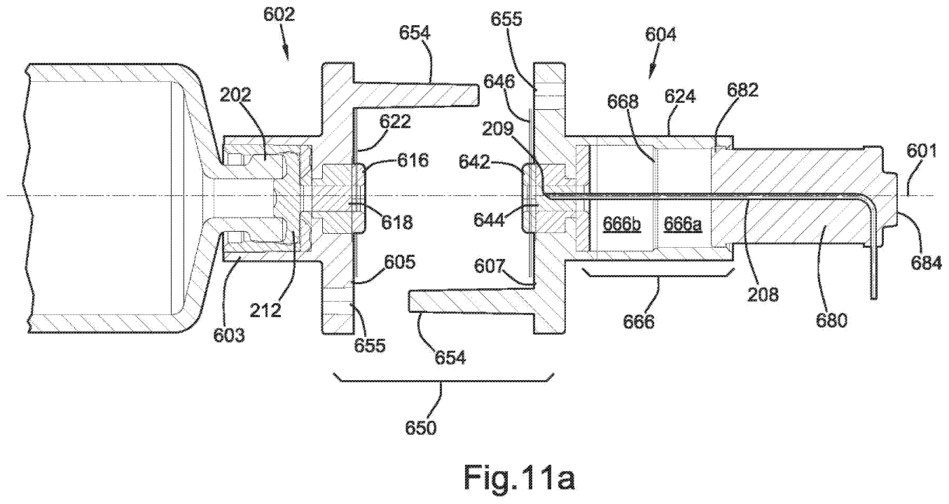

With reference to FIGS. 10-11c, the second connection assembly 604 includes a body 624 configured to slidably receive the transfer needle 208 therein. The body 624 defines a chamber 666 therein. The transfer needle 208 is movable within the chamber 666 as will be described further below. A second coupling surface 607 is defined on the body 624 and is configured to be placed adjacent to, or in contact with, the first coupling surface 605 of the first connection assembly 602. An opening 644 is defined on the body 624 and is in fluid communication with the chamber 666. The second connection assembly 604 may include a seal 642 thereon. The seal 642 can be on, in, or adjacent to the opening 644. The seal 642 can include an O-ring, a gasket, or another suitable seal. The seal 642 can form a sealing interface on the second connection assembly 604 and can be contacted with one or more components of the first connection assembly 602, such as the body 603 and/or the seal 616.

The connection system 600 can include a coupler or a coupling assembly 650 configured to connect and secure the first and second connection assemblies 602 and 604. In some aspects, the coupling assembly 650 may be similar to, or substantially the same as, any of the connection mechanisms described elsewhere in this application, such as the first clip mechanism 450. Referring to FIGS. 11a-11c, the coupling assembly 650 may include a protrusion 654 on one of the first and second connection assemblies 602 and 604 that is configured to engage with a corresponding receptacle 655 on the other of the first and second connection assemblies 602 and 604. The receptacle 655 may be a notch, a groove, or the like. The engagement between the protrusion 654 and the receptacle 655 may be that of a snap fit, friction fit, interference fit, or another suitable mechanism. It will be appreciated that at least one of the protrusion 654 and the receptacle 655 may include one or more components thereon configured to facilitate engagement therebetween, such as ridges, teeth, notches, and the like. Each of the first and second connection assemblies 602 and 604 may include a plurality of protrusions 654 and respective corresponding receptacles 655. In some aspects, all of the protrusions 654 may be disposed on one of the first and second connection assemblies 602 and 604, while all of the corresponding receptacles 655 may be disposed on the other of the first and second connection assemblies 602 and 604. Alternatively, each of the first and second connection assemblies 602 and 604 may include at least one of the protrusions 654 and at least one of the receptacles 655, with the other of the first and second connection assemblies 602 and 604 includes the corresponding at least one of the receptacles 655 and the at least one protrusions 654. Referring to the exemplary embodiments depicted in FIGS. 9-11c, the first connection assembly 602 may include one protrusion 654 and one receptacle 655. Similarly, the second connection assembly 604 may also include one protrusion 654 and one receptacle 655. The protrusion 654 of the first connection assembly 602 can engage with the receptacle 655 on the second connection assembly 604, and the protrusion 654 on the second connection assembly 604 can engage with the receptacle 655 on the first connection assembly 602.

During assembly of the connection system 600, the first connection assembly 602 may be moved toward the second connection assembly 604 along an axis 601 (see FIGS. 10-11c). It will be appreciated that the second connection assembly 604 can instead be moved toward the first connection assembly 602, or that both the first and second connection assemblies 602 and 604 may be moved towards one another along the axis 601.

The transfer needle 208 is movable within the second connection assembly 604 and can be moved toward and/or away from the first connection assembly 602 along the axis 601. With continued reference to FIGS. 10-11c, the transfer needle 208 may be disposed on, or in, a needle hub 680 that is configured to be slidably movable within the chamber 666 of the second connection assembly 604. The transfer needle 208 may be fixedly secured to the needle hub 680, such that when the needle hub 680 is moved toward the opening 644 of the second connection assembly 604, the transfer needle 208 is also moved toward the opening 644. Disposing the transfer needle 208 within the needle hub 680 can facilitate proper orientation and angling of the transfer needle 208 during coupling of the first and second connection assemblies 602 and 604 and during engagement of other components as described elsewhere in this application. Securing the transfer needle 208 to the needle hub 680 that is slidably movable within the second connection assembly 604 allows for the needle hub 680 to be kept in the desired orientation during movement of the second connection assembly 604, and thus keep the transfer needle 208 in the desired position relative to the opening 644 and/or the first connection assembly 602. Such an arrangement can simplify the aiming of the transfer needle 208 relative to the openings 644 and 618 and to prevent the transfer needle 208 from being directed toward or through the openings 644 and/or 618 at an undesired angle, which could otherwise cause damage to the transfer needle 208 or any of the related components of the connection system 600.

The needle hub 680 (and, thus, the transfer needle 208) may be configured to be disposed at different positions relative to the rest of the connection system 600 based on the configuration of the first and second connection assemblies 602 and 604. In a first configuration, such as shown in FIG. 11a, the needle hub 680 can be in a first position within the chamber 666. When the needle hub 680 is in the first position, the transfer needle 208 is within the second connection assembly 604 and does not extend out of the opening 644. A needle tip 209 of the transfer needle 208 is fully within the second connection assembly 604 and is spaced from the septum 212. When the connection assembly 600 is in the first configuration, the first and second connection assemblies 602 and 604 are spaced from one another and are not coupled together via the coupling assembly 650.

In a second configuration, the first and second connection assemblies 602 and 604 can be brought closer together and coupled via the coupling assembly 650 described above. The first and second connection assemblies 602 and 604 can be fixedly secured to one another, and a seal can be formed between the opening 618 of the first connection assembly 602 and the opening 644 of the second connection assembly 604. The seal may be formed via contact between the seal 616 of the first connection assembly 602 and the seal 642 of the second connection assembly 604. The seal can be formed via contact of the seal 616 and/or the seal 642 with the body 624 of the second connection assembly 604 and/or the body 603 of the first connection assembly 602, respectively.

When the connection system 600 is in the second configuration, the needle hub 680 can be moved into a second position along the axis 601, such as shown in FIG. 11b. The transition from the first position to the second position can include slidably moving the needle hub 680 (and, thus, the transfer needle 208) along the axis 601 toward the opening 644. When the needle hub 680 is in the second position, the transfer needle 208 can be moved through the opening 644 and toward or into the opening 618 of the first connection assembly 602. In the second position, the needle tip 209 is outside of the second connection assembly 604 and can be at least partly inside the first connection assembly 602. The needle tip 209 is still spaced away from, and does not pierce, the septum 212 in this second position.

The connection system 600 can be transitioned into a third configuration from the second configuration. When the connection system 600 is in the third configuration, the needle hub 680 can be in a third position. The needle hub 680 can be transitioned from the second position to the third position by being slidably moved along the axis 601 toward the opening 644. In the third position, the needle tip 209 is moved toward and through the septum 212, such that the needle tip 209 pierces the septum 212 and enters the medicament containers 202. At this point, a fluid connection can be established between the medicament containers 202 and the transfer needle 208.

The specific position, arrangement, and orientation of the needle hub 680 relative to the second connection assembly 604 can depend on the interaction of the needle hub 680 and the chamber 666. Referring again to FIGS. 10-11c, the chamber 666 may vary in size and/or shape along the axis 601. In some aspects, the chamber 666 can include a first segment 666a and a second segment 666b adjacent to the first segment 666a. The second segment 666b may be between the first segment 666a and the opening 644. The first and second segments 666a and 666b may have different dimensions. In some aspects, the cross-sectional dimension (e.g., a diameter 667a) of the first segment 666a may be smaller than the cross-sectional dimension (e.g., a diameter 667b) of the second segment 666b. The different cross-sectional dimensions can facilitate positioning of the needle hub 680 within the chamber 666 at each of the first, second, and third positions. For example, when the needle hub 680 is in the first position, the needle hub 680 contacts the internal walls of the chamber 666 within the first segment 666a. When the needle hub 680 is in the second and/or third positions, at least a portion of the needle hub 680 is disposed in the chamber 666 within the second segment 666b. An engagement between the needle hub 680 and the walls of the chamber 666 in the first segment 666a may define a tighter fit between the needle hub 680 and the body 624, thus requiring a greater force to slidably move the needle hub 680 from the first position to the second position. Subsequently, the engagement between the needle hub 680 and the walls of the chamber 666 of the second segment 666b may define a looser fit between the needle hub 680 and the body 624 relative to that of the first segment 666a, thus requiring less force to slidably move the needle hub 680 from the second position to the third position relative to the force required to move the needle hub 680 from the first position to the second position. The difference in required forces can help secure the needle hub 680 within the first position to decrease the risk of inadvertent movement of the needle hub 680 out of the first position.

The needle hub 680 may include one or more features thereon configured to contact, or otherwise engage with, the inner walls of the chamber 666 when the needle hub 680 is in the first, second, and/or third positions. Referring again to FIGS. 10-11c, the needle hub 680 may include a protrusion 682 thereon, for example, at one end thereof closest to the needle tip 209. The protrusion 682 may include a circumferential ridge extending partially or entirely around the needle hub 680. In some aspects, the needle hub 680 can include a plurality of protrusions 682, for example, being radially disposed around the needle hub 680. With specific reference to FIG. 10, a cross-sectional measurement (e.g., a diameter 683a) of the needle hub 680 at the protrusion 682 may be greater than the cross-sectional measurement (e.g., a diameter 683b) of the needle hub 680 without the protrusion 682. The diameter 667a of the first segment 666a of the chamber 666 can be greater than the diameter 683b of the needle hub 680, but less than the diameter 683a of the needle hub 680. As such, when the portion of the needle hub 680 containing the protrusion 682 is within the first segment 666a, a greater friction fit can be established between the needle hub 680 and the body 624 of the second connection assembly 604. The diameter 667b of the second segment 666b can be greater than the diameter 683a of the needle hub 680, and thus, when the protrusion 682 of the needle hub 680 is within the second segment 666b, a lesser friction fit can be established between the body 624 and the needle hub 680 compared to when the protrusion 682 is in the first segment 666a. It will be appreciated that the body 624 and/or the needle hub 680 should be formed of a suitable material that is configured to deform and/or deflect sufficiently during the established contact between the needle hub 680 and the body 624 such that the needle hub 680 can be moved within the chamber 666 upon application of a threshold force to the needle hub 680 and/or to the second connection assembly 604.

The body 624 of the second connection assembly 604 may include a ledge 668 extending into the chamber 666. The ledge 668 may separate the first and second segments 666a and 666b and may be between the first and second segments 666a and 666b. The ledge 668 may extend partially or entirely circumferentially around the interior of the body 624 that defines the chamber 666. In use, the ledge 668 may be configured to be contacted by the protrusion 682 of the needle hub 680 when the needle hub 680 is being moved from the first position to the second position and/or form the second position to the third position. As seen in the figures, in some aspects, the ledge 668 may be sloped toward the opening 644, such that the protrusion 682 of the needle hub 680 can slidably move along the ledge 668 during movement of the needle hub 680 when the protrusion 682 is moved from the first segment 666a to the second segment 666b. Such a sloped arrangement can facilitate slidable movement of the needle hub 680 toward the opening 644. The sloped arrangement can further prevent or hinder movement of the needle hub 680 away from the opening 644 when the protrusion 682 is within the second segment 666b by requiring a greater force to be applied to the needle hub 680 to cause the protrusion 682 to slide along the ledge 668 into the first segment 666a from the second segment 666b than the force that is needed to be applied to the needle hub 680 to cause the protrusion 682 to slide along the ledge 668 into the second segment 666b from the first segment 666a.

It will be appreciated that the connection system 600 can include a first and second flexible barrier, such as the first and second flexible barriers 422 and 446 described previously. It should also be understood that the included flexible barriers can be formed of a suitable size and shape to accommodate the dimensions of the individual components described above with respect to the connection system 600. An exemplary first flexible barrier 622 and second flexible barrier 646 are depicted in FIG. 11a. The user can remove the first and second flexible barriers 622 and 646 upon engagement of the first and second connection assemblies 602 and 604 to result in a sterile fluid communication between the first and second connection assemblies 602, and specifically between the medicament container 202 and the transfer needle 208. The connection system 600 may include similar or identical flexible barriers as those described previously, and the description of the first and second flexible barriers 422 and 446 and their respective uses and mechanisms is incorporated herein with respect to the first and second flexible barriers 622 and 646 and to the connection system 600.

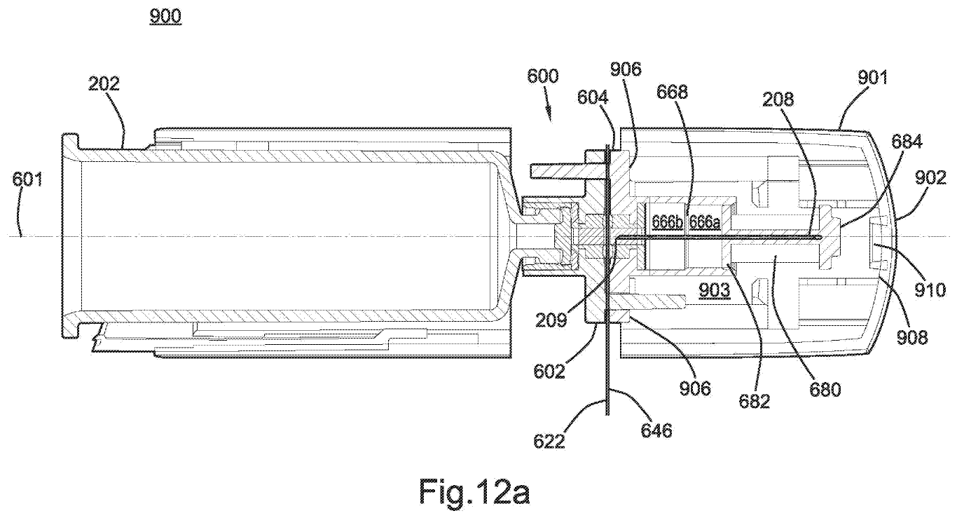

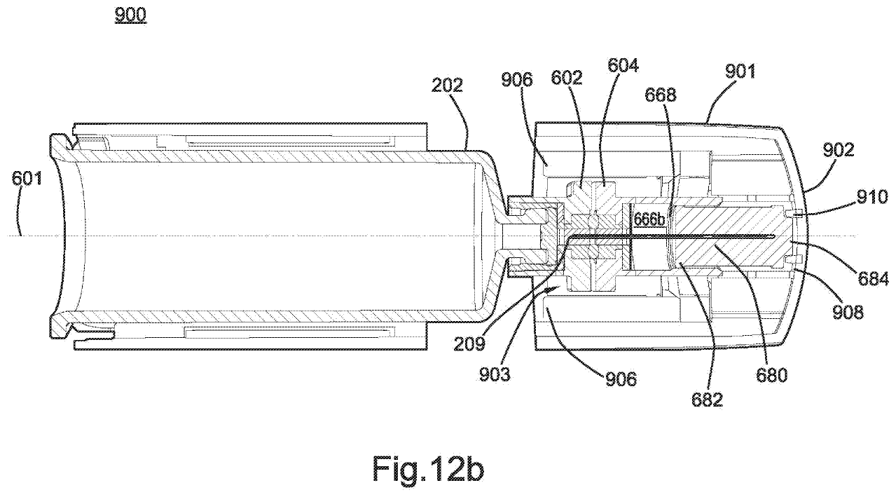

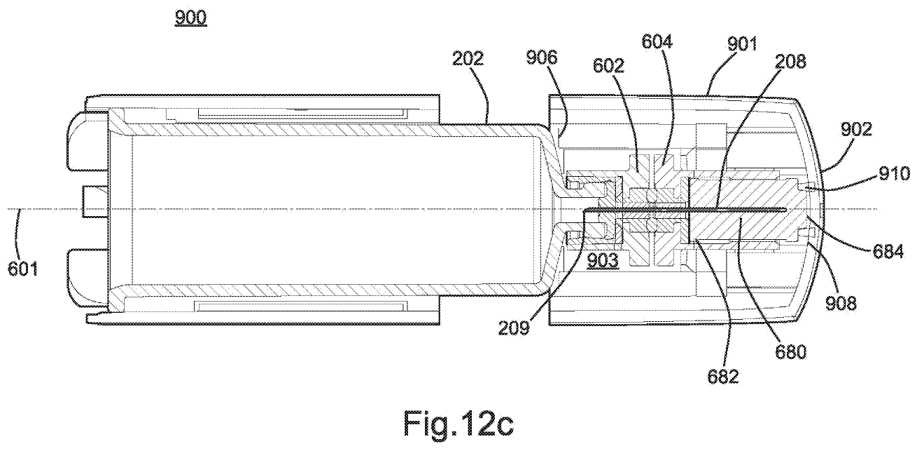

The connection system 600 may be utilized in an injection device, such as the wearable injection system device 200 described previously or another suitable injection device. A portion of an exemplary injection device 900 is depicted in FIGS. 12a-12c. It will be appreciated that any suitable injection device can be utilized with the connection system 600, and this disclosure is not intended to limit the application of the connection system 600 to a particular injection device. The injection device 900 can include a door 901 configured to receive the connector system 600 and/or the medicament container 202 therein. The door 901 can be similar to, or the same as, the door 222 described previously or another suitable door structure.

The door 901 can define a receptacle 903 therein configured to receive the connector system 600 and/or the medicament container 202. At least a portion of the receptacle 903 can be dimensioned to receive the connector system 600 therein. In some aspects, the receptacle 903 can be dimensioned or keyed such that the connector system 600 is receivable therein only in a predetermined orientation. The connector system 600 may be designed such that the transition from the first configuration to the second configuration, or form the second configuration to the third configuration, is not possible unless the connector system 600 is properly situated within the receptacle 903. The keyed arrangement of the receptacle 903 can thus prevent inadvertent or premature connection of components of the connector system 600 by ensuring that the individual components are actively positioned according to the required orientation to be received within the receptacle 903.

During use, the connection system 600 can be moved along the axis 601 toward and into the receptacle 903 toward a first end 902 of the door 901. The door 901 can include one or more ribs 906 extending from an inner surface of the door 901 into the receptacle 903. The ribs 906 can define the keyed arrangement of the receptacle 903. The connection system 600 can have at least a first insertion orientation and a second insertion orientation. When the connection system 600 is in the first insertion orientation and is moved into or through the receptacle 903 toward the first end 902, the connection system 600 can contact the one or more ribs 906. This contact can preclude the connection system 600 from further movement toward the first end 902. When the connection system 600 is in the second insertion orientation, the connection system 600 can be moved toward the first end 902 without substantially contacting the one or more ribs 906, and is thus not blocked by the ribs 906 from further movement along the axis 601. The second insertion orientation can be rotationally offset from the first insertion orientation around the axis 601. To transition the connection system 600 from the first insertion orientation to the second insertion orientation, the connection system 600 may be rotated about the axis 601 by a predetermined distance. In some aspects, the connection system 600 may need to be rotated by approximately 15 degrees, approximately 30 degrees, approximately 45 degrees, approximately 60 degrees, approximately 90 degrees, approximately 120 degrees, or approximately 180 degrees relative to the first insertion orientation to be placed into the second insertion orientation. Referring to the exemplary embodiment depicted in FIGS. 12a-12c, the second insertion orientation of the connection system 600 is approximately 90 degrees rotationally offset from the first insertion orientation. In some aspects, rotation of the connection system 600 may be limited to only one direction around the axis 601. Alternatively, the connection system 600 may be permitted to be rotated in either opposing direction around the axis 601 to transition between the first and second insertion orientations.

Referring to FIG. 12a, for example, the connection system 600 is depicted in the first insertion orientation. As such, when the connection system 600 is moved along the axis 601 toward the first end 902, the connection system 600 contacts the ribs 906 of the door 901 and is precluded from moving past the ribs 906. The needle hub 680 is in its first position when the connection system 600 is in the first insertion orientation. Prior to the first and second connection assemblies 602 and 604 being connected to each other, the first and second flexible barriers 622 and 646 are still affixed to the first and second connection assemblies 602 and 604, respectively. When the first and second connection assemblies 602 and 604 are coupled as shown in FIG. 12a, the first and second flexible barriers 622 and 646 can be removed, thus placing the opening 618 of the first connection assembly 602 in fluid communication with the opening 644 of the second connection assembly 604.

Turning to FIG. 12b, components of the connection system 600 are shown having been rotated approximately 90 degrees about the axis 601 into the second insertion orientation. In the second insertion orientation, the connection system 600 can be moved further along the axis 601 toward the first end 902. The connection system 600 clears the ribs 906 when axially translated toward the first end 902 in the second insertion orientation.

As the connection system 600 moves toward the first end 902, the needle hub 680 can contact a wall 908 of the door 901 at the first end 902. The wall 908 can prevent further axial translation of the needle hub 680. As the rest of the connection system 600 is translated along the axis 601 toward the first end 902, the body 624 of the second connection assembly 604 can slidably move over the needle hub 680, such that the chamber 666 is slidably moved relative to the needle hub 680. This movement causes the needle hub 680 to be moved from its first position to its second position. It will be appreciated that the movement between the needle hub 680 and the body 624 is relative, and this description is not limited to one component moving while the other being stationary, or vice versa.

Referring to FIG. 12c, the connection system 600 is shown in its third configuration. The connection system 600 is depicted having been moved further along the axis 601 toward the first end 902. Because the needle hub 680 is precluded from further axial movement, further translation of the rest of the connection system 600 relative to the needle hub 680 causes the needle hub 680 to be moved into its third position, where the needle tip 209 contacts and pierces the septum 212 of the medicament container 202. The connection system 600 can be moved toward the first end 902 until the body 624 of the second connection assembly 604 axially contacts the needle hub 680 and is thus precluded from further axial movement.

In some aspects, the needle hub 680 can include a keyed projection 684 thereon that is configured to engage with a corresponding keyed receptacle 910 defined on the wall 908. The cross-sectional geometry of the keyed projection 684 can be complementary to the cross-sectional geometry of the keyed receptacle 910, such that the needle hub 680 can be received into the keyed receptacle 910 only when in the desired rotational orientation relative to the axis 601. The engagement between the keyed receptacle 910 and the keyed projection 684 can help prevent rotation of the needle hub 680 and/or the rest of the connection system 600 after the needle hub 680 contacts the wall 908. This can decrease the chance of incorrect angling of the transfer needle 208 or damage to the transfer needle 208 due to undesired movement of the needle hub relative to the wall 908.

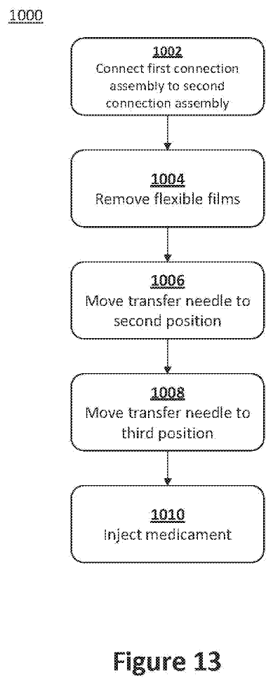

FIG. 13 depicts a flow chart of an exemplary method 1000 of connection and use of the injection device 900 with a connection system 600 described above. In step 1002, the first connection assembly 602 is engaged with the second connection assembly 604. This step can include securing the first and second connection assemblies 602 and 604 together via the coupling assembly 650. Step 1002 can include moving the one or more protrusions 654 towards and into engagement with their respective one or more receptacle 655.

In step 1004, the one or more flexible barriers 622 and 646 can be removed from the first and second connection assemblies 602 and 604, respectively. The first flexible barrier 622 can be removed before the second flexible barrier 646, or vice versa. In some aspects, both flexible barriers 622 and 646 can be removed simultaneously. The seals 616 and 642 help form an aseptic seal between the first and second connection assemblies 602 and 604 once the flexible barriers 622 and 646 are removed. Once the flexible barriers 622 and 646 are removed, the opening 618 in the first connection assembly 602 is in fluid communication with the opening 644 of the second connection assembly 604.

In step 1006, the transfer needle 208 can be moved from its first position to its second position, where the needle tip 209 is moved towards the first connection assembly 602. The needle tip 209 can be moved into the opening 618 of the first connection assembly 602. At this stage, the transfer needle 208 is still spaced from the septum 212 and the medicament container 202. The transfer needle 208 can be moved by contacting the needle hub 680 with the wall 908 of the door 901 of the injection device described above and causing the body 624 of the second connection assembly 604 to slidably move over the needle hub 680, thus moving the opening 644 toward the needle tip 209.

In some aspects, prior to step 1006, the connection system 600 may have to be oriented properly to be permitted to move within the receptacle 903 of the injection device 900 as described above. The one or more protrusions or ribs 906 can be disposed in the receptacle 903 such that the connection system 600 is blocked from being translated toward the wall 908 when the connection system 600 is in the first insertion orientation. Accordingly, prior to being able to complete step 1006, the connection system 600 may need to be rotated from the first insertion orientation to the second insertion orientation as described above.

In step 1008, the transfer needle 208 can be moved to its third position, where the needle tip 209 is moved towards the septum 212 and contacts and pierces the septum 212. After the needle tip 209 has pierced the septum 212, the transfer needle 208 is in fluid communication with the interior of the medicament container 202. A medicament inside the medicament container 202 can then be moved from the medicament container 202 into the transfer needle 208.

In step 1010, the medicament can be delivered from the medicament container 202, through the transfer needle 208, to a hypodermic needle (e.g., the hypodermic needle 104), and to the desired injection site (e.g., the skin 106). Step 1010 can include actuating an actuation mechanism configured to

The method 1000 can further include a step of positioning the injection device 900 at the desired injection site.

The method 1000 can also include moving the hypodermic needle towards and/or into the injection site. The hypodermic needle can be moved via a mechanism configured to cause the hypodermic needle to be moved to the desired location (e.g., by pressing the actuation button 220).

The method 1000 can also include actuating securing the injection device 900 to the user's skin 106.

The methods and processes described herein are not limited to any particular sequence, and the steps relating thereto may be performed in other sequences that are appropriate. For example, described steps may be performed in an order other than that specifically described, or multiple steps may be combined in a single step. The described steps may be performed in serial, in parallel, or in some other manner. Steps may be added to or removed from the depicted methods. One or more steps, or sequence of steps, can be repeated. Unless otherwise expressly stated, the described methods should not be construed as requiring that its operations be performed in a specific order.

The preceding detailed description describes systems and methods for ensuring sterility of an on-body injection device (e.g., the injection devices 100 or 900). However, the skilled person will understand that the invention is not limited to use in connection with the exemplary on-body device described here. Rather, one or more benefits associated with the present invention may be implemented in connection with other drug delivery devices, as will be apparent to the skilled person in light of the preceding detailed description.

The term “comprising” should be interpreted as meaning “including but not limited to”, such that it does not exclude the presence of features not listed.

The embodiments described and shown in the accompanying drawings above are provided as examples of ways in which the invention may be put into effect and are not intended to be limiting on the scope of the invention. Modifications may be made, and elements may be replaced with functionally and structurally equivalent parts, and features of different embodiments may be combined without departing from the disclosure.

1. A connection system for use with an injection device, the connection system comprising:
a first connection assembly, a second connection assembly, and a coupling assembly configured to secure the first and second connection assemblies together,
the first connection assembly comprising:
a first body configured to connect to a medicament container, the first body defining a first opening therethrough configured to be in fluid communication with the medicament container;
a first coupling surface on the first body;
a first seal adjacent to the first coupling surface; and
a first flexible barrier disposed on the first coupling surface, such that the first opening is closed by the first flexible barrier; and
the second connection assembly comprising:
a second body configured to receive a needle therein, the second body defining a second opening therethrough;
a second coupling surface on the second body, the second coupling surface being configured to be placed adjacent to the first coupling surface;
a second flexible barrier disposed on the second coupling surface, such that the second opening is closed by the second flexible barrier; and
a second seal adjacent to the second coupling surface,
wherein, when the first and second connection assemblies are secured together, an insertion axis is defined parallel to the needle and extending through the first and second openings of the first and second connection assemblies,
wherein, when the first and second connection assemblies are secured together, the first and second flexible barriers are configured to be removed, such that the first and second openings fluidly communicate with each other,
wherein the needle is configured to be moved through the second opening and the first opening and into the medicament container, such that the needle is in fluid communication with the medicament container, and
wherein an aseptic connection is formed between the first and second connection assemblies.

2. The connection system of claim 1, wherein the needle is configured to pierce a septum of the medicament container so as to create the fluid communication between the needle and the medicament container.

3-4. (canceled)

5. The connection system of claim 1, wherein the coupling assembly includes a protrusion on one of the first and second bodies and a receptacle on the other of the first and second bodies, the receptacle being configured to receive the protrusion therein when the first and second connection assemblies are moved towards each other, and wherein, when the protrusion is in the receptacle, movement of the first and second connection assemblies away from each other is precluded.

6. (canceled)

7. The connection system of claim 1, wherein:
the needle is configured to be movable between a first position, a second position, and a third position,
when the needle is in the first position, a tip of the needle is entirely within the second connection assembly and does not extend out of the second opening;
when the needle is in the second position, the tip of the needle is disposed within the first opening of the first connection assembly; and
when the needle is in the third position, the tip of the needle is configured to be in the medicament container.

8. The connection system of claim 1, wherein the needle is disposed on a needle hub movable within a chamber defined by the second body of the second connection assembly.

9. The connection system of claim 8, wherein the chamber has a first segment having a first diameter and a second segment having a second diameter different from the first diameter, and wherein the friction between the needle hub and the first segment is greater than the friction between the needle hub and the second segment.

10. The connection system of claim 8, wherein the needle hub defines a keyed protrusion thereon configured to be received into a corresponding keyed receptacle on an injection device.

11. An injection device for providing a medicament to a user, the injection device comprising:
a housing having an injection needle configured to be moved to an injection site;
a medicament container disposed in the housing, the medicament container being configured to receive the medicament therein;
a transfer needle configured to be placed into fluid communication with the injection needle and the medicament container, such that the medicament can be moved from the medicament container to the injection needle through the transfer needle; and
the connection system of claim 1 configured to form an aseptic connection between the transfer needle and the medicament container.

12-14. (canceled)

15. The injection device of claim 11, wherein the housing defines a receptacle configured to receive the medicament container and the connection system therein.

16. The injection device of claim 15, wherein the connection system is configured to have a first rotational orientation and a second rotational orientation angularly displaced from the first rotational orientation, and
wherein the housing includes a first keyed feature extending into the receptacle, the receptacle being configured to preclude movement of the connection system into the receptacle when the connection system is in the first rotational orientation, and to allow movement of the connection system into the receptacle when the connection system is in the second rotational orientation.

17. The injection device of claim 11, wherein the connection system comprises a coupling assembly configured to secure the first and second connection assemblies together.

18. The injection device of claim 11, wherein the medicament container includes a septum thereon that provides a liquid seal between the medicament in the medicament container and the connection system, wherein the septum is configured to be pierced by the transfer needle such that the fluid communication is formed between the transfer needle and the medicament container.

19. The injection device of claim 11, wherein the housing includes a movable door configured to be opened and closed, the medicament container being receivable onto the movable door.

20. The injection device of claim 11, further comprising an activation button assembly configured to cause movement of the injection needle from a first position, in which the injection needle is spaced from the injection site, to a second position, in which the needle contacts the injection site.

21-31. (canceled)

32. A connection system for creating an aseptic connection between a container and a delivery conduit of an injection device, the connection system comprising:
a first connection assembly comprising:
a first engagement body having a first end that includes a first opening and is configured to form a seal with a septum of a container, a second end having a second opening, a first cavity between the first and second openings, and a first engagement surface at the second end of the first engagement body; and
a first flexible barrier configured to form a seal with the second opening; and
a second connection assembly comprising:
a second engagement body having a first end that includes a third opening, a second end that includes a fourth opening, a second cavity between the third and fourth openings, and a second engagement surface at the second end of the second engagement body; and
a needle operably connected to the second engagement body; and a second flexible barrier configured to form a seal with the fourth opening,
wherein the first engagement surface further comprises a first sealing interface configured to form a seal with a second sealing interface on the second engagement surface, and
wherein the connection system is configured such that the needle is moveable relative to the fourth opening of the second engagement body from a first position where the needle is spaced from the third opening, to a second position where the needle extends through the third opening.

33. (canceled)

34. The connection system of claim 32, wherein the needle is translatable relative to the second engagement surface, to thereby enable the needle to pass into the first cavity once the seal between the first and second sealing interfaces has been created.

35-51. (canceled)

52. A container filling system comprising:
a container;
a bulk storage container for filling the container with a medicament, the bulk storage container having an interior volume that is larger than that of the container; and
the connection system of claim 32,
wherein the bulk storage container is attached to one of the first and second connection assemblies, and the container is connected to the other of the first and second connection assemblies.

53-54. (canceled)
